(19) United States
(12) Patent Application Publication
(10) Pub. No.: US 2023/0301599 A1
(43) Pub. Date: Sep. 28, 2023

(54) Method and System for Detection of Inflammatory Conditions

(71) Applicant: Emerald Innovations Inc., Cambridge, MA (US)

(72) Inventors: Rumen Hristov, Cambridge, MA (US); Hariharan Rahul, Boston, MA (US); Shichao Yue, Somerville, MA (US); Yuqing Ai, Seattle, WA (US); Bruce Maggs, Chapel Hill, NC (US); Dina Katabi, Boston, MA (US)

(21) Appl. No.: 18/188,160

(22) Filed: Mar. 22, 2023

Related U.S. Application Data
(60) Provisional application No. 63/269,723, filed on Mar. 22, 2022.

(51) Int. Cl. A61B 5/00; G16H 50/20

(52) U.S. Cl. CPC A61B 5/7264; A61B 5/7282; G16H 50/20

(57) ABSTRACT

A wireless method for predicting an inflammation state of a person under observation, comprising: (a) transmitting frequency-modulated continuous-wave (FMCW) wireless signals from one or more transmitting antennas; (b) receiving reflected FMCW wireless signals with one or more receiving antennas, at least some of the reflected FMCW wireless signals being reflected from the person; (c) repeating steps (a) and (b) continuously while the person is under observation; (d) producing reflected FMCW wireless data based on the reflected FMCW wireless signals; (e) providing the reflected FMCW wireless data as an input to a trained machine-learning (ML) model, the trained ML model having been trained with ground-truth data that represents ground-truth inflammation states and ground-truth reflected FMCW wireless data of one or more subjects with respect to time; and (f) predicting, with the trained ML model, whether the person under observation is in an inflamed state or a non-inflamed state.

### CROSS-REFERENCE TO RELATED APPLICATIONS

This application claims priority to U.S. Provisional Application No. 63/269,723, titled “Method and System for Detection of Inflammatory Conditions,” filed on Mar. 22, 2022, which is hereby incorporated by reference.

### TECHNICAL FIELD

This application relates generally to motion tracking using wireless signals.

### BACKGROUND

People with inflammatory diseases, such as Crohn's disease or Lupus, often experience symptoms that can come and go. During a flare-up, the symptoms of the disease reappear and the health of the person worsens. Later, a person may have a remission, which means that the symptoms improve or disappear for a period of time. During periods of flare-up, people exhibit the usual signs of inflammation: redness, heat, swelling, pain, loss of function, etc. If, in the case of a flare-up, the first signs of inflammation are detected early, one can intervene and mitigate the worsening of the symptoms and possibly bring the person back to remission more quickly. Current methods for detecting inflammation include scheduled doctor visits, where a doctor performs blood tests, stool tests, general physical examinations, or other tests. The existing methods are not efficient, and they are often performed after inflammation levels have already increased significantly.

### SUMMARY

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Without limiting the scope of the claims, some of the advantageous features will now be summarized. Other objects, advantages and novel features of the disclosure will be set forth in the following detailed description of the disclosure when considered in conjunction with the drawings, which are intended to illustrate, not limit, the invention.

An aspect of the invention is directed to a wireless method for predicting an inflammation state of a person under observation, comprising: (a) transmitting frequency-modulated continuous-wave (FMCW) wireless signals from one or more transmitting antennas; (b) receiving reflected FMCW wireless signals with one or more receiving antennas, at least some of the reflected FMCW wireless signals being reflected from the person partially or fully; (c) repeating steps (a) and (b) continuously while the person is under observation; (d) producing reflected FMCW wireless data based on the reflected FMCW wireless signals; (e) providing the reflected FMCW wireless data as an input to a trained machine-learning (ML) model, the trained ML model having been trained with ground-truth inflammation that represents ground-truth inflammation states of one or more subjects with respect to time and with ground-truth reflected FMCW wireless data of the one or more subjects with respect to time; and (f) predicting, with the trained ML model, whether the person under observation is in an inflamed state or in a non-inflamed state.

In one or more embodiments, the trained ML model includes a trained neural network. In one or more embodiments, the trained ML model includes a trained recurrent neural network, a trained feedforward neural network, or a trained convolutional neural network.

In one or more embodiments, step (d) comprises converting a discrete time period of raw reflected FMCW wireless data into a three-dimensional reflected wireless signal map, the three-dimensional reflected wireless signal map including a plurality of voxels that correspond to a respective physical location in a room in which the person is located, each voxel having a respective numerical value that represents a magnitude and a phase of the reflected FMCW wireless signal(s) that was/were reflected from the respective physical location, and step (e) comprises providing the three-dimensional reflected wireless signal map as the input to the trained ML model, the trained ML model having been trained with ground-truth three-dimensional reflected wireless signal maps with respect to time. In one or more embodiments, the input to the trained ML model further includes the raw reflected FMCW wireless data, and the trained ML model was trained with ground-truth raw reflected FMCW wireless data with respect to time.

Another aspect of the invention is directed to a wireless method for predicting an inflammation state of a person under observation, comprising: (a) transmitting frequency-modulated continuous-wave (FMCW) wireless signals from one or more transmitting antennas; (b) receiving reflected FMCW wireless signals with one or more receiving antennas, at least some of the reflected FMCW wireless signals being reflected from the person partially or fully; (c) repeating steps (a) and (b) continuously while the person is under observation; (d) producing raw reflected FMCW wireless data from the reflected FMCW wireless signals; (e) converting a plurality of discrete time periods of the raw reflected FMCW wireless data into respective three-dimensional reflected wireless signal maps, each three-dimensional reflected wireless signal map including a plurality of voxels that correspond to a respective physical location in a room in which the person under observation is located, each voxel having a respective numerical value that represents a magnitude and a phase of the reflected FMCW wireless signal(s) that was/were reflected from the respective physical location; (f) determining a health indicator of the person under observation based on a plurality of three-dimensional reflected wireless signal maps; (g) determining one or more quantifiable health metrics related to the health indicator; (h) providing the quantifiable health metric(s) as an input to a trained machine-learning (ML) model, the trained ML model having been trained with ground-truth inflammation that represents ground-truth inflammation states of one or more subjects with respect to time and with ground-truth quantifiable health metric data of the one or more subjects with respect to time; and (i) predicting, with the trained ML model, whether the person under observation is in an inflamed state or in a non-inflamed state.

In one or more embodiments, the trained ML model includes a trained ML classifier. In one or more embodiments, the trained ML classifier includes a support vector classifier or a support vector machine.

In one or more embodiments, the input to the trained ML model further includes the respective three-dimensional reflected wireless signal maps, and the trained ML model was trained with ground-truth three-dimensional reflected wireless signal maps with respect to time. In one or more embodiments, the input to the trained ML model further includes the raw reflected FMCW wireless data, and the trained ML model was trained with ground-truth raw reflected FMCW wireless data with respect to time.

In one or more embodiments, the health indicator includes a respiration of the subject under observation, the quantifiable health metric(s) include a respiration rate of the subject under observation and/or an average respiration rate of the subject under observation, and the ground-truth quantifiable health metric data includes a ground-truth respiration rate of the one or more subjects with respect to time and/or an average ground-truth respiration rate of the one or more subjects with respect to time. In one or more embodiments, the health indicator is a first health indicator, the quantifiable health metric(s) is/are first quantifiable health metric(s), and the method further comprises: determining a second health indicator of the person under observation based on the three-dimensional wireless signal maps; determining one or more second quantifiable health metrics related to the second health indicator; and providing the first and second quantifiable health metric(s) as the input to the trained ML model, wherein the ground-truth quantifiable health metric data used to train the trained ML model is related to the first and second quantifiable health metrics.

In one or more embodiments, the second health indicator includes a physical location of the subject under observation, the second quantifiable health metric(s) include a gate speed of the subject under observation and/or an average gate speed of the subject under observation, and the ground-truth quantifiable health metric data further includes a ground-truth gate speed of the one or more subjects with respect to time and/or an average gate speed of the one or more subjects with respect to time.

In one or more embodiments, the method further comprises sending an output signal to a device or an account controlled by the subject under observation, the output signal including whether the person under observation is in the inflamed state or in the non-inflamed state.

Another aspect of the invention is directed to a wireless-tracking system comprising: one or more transmitting antennas configured to transmit frequency-modulated continuous-wave (FMCW) wireless signals; one or more receiving antennas configured to receive reflected FMCW wireless signals, at least some of the reflected FMCW wireless signals being reflected, partially or fully, from a person under observation; a processor circuit electrically coupled to the one or more transmitting antennas and the one or more receiving antennas; a power supply electrically coupled to the processor circuit; and non-transitory computer-readable memory in electrical communication with the processor circuit, the non-transitory computer-readable memory storing computer-readable instructions that, when executed by the processor circuit, cause the processor circuit to: produce reflected FMCW wireless data based on the reflected FMCW wireless signals; provide the reflected FMCW wireless data as an input to a trained machine-learning (ML) model, the trained ML model having been trained with ground-truth inflammation that represents ground-truth inflammation states of one or more subjects with respect to time and with ground-truth reflected FMCW wireless data of the one or more subjects with respect to time; and predict, with the trained ML model, whether the person under observation is in an inflamed state or in a non-inflamed state.

In one or more embodiments, the one or more transmitting antennas comprise a plurality of the transmitting antennas, the one or more receiving antennas comprise a plurality of the receiving antennas, and the transmitting and receiving antennas are arranged along two orthogonal axes. In one or more embodiments, the transmitting antennas and the receiving antennas are evenly spaced along the two orthogonal axes.

In one or more embodiments, the computer-readable instructions that, when executed by the processor circuit, further cause the processor circuit to: convert a discrete time period of raw reflected FMCW wireless data into a three-dimensional reflected wireless signal map, the three-dimensional reflected wireless signal map including a plurality of voxels that correspond to a respective physical location in a room in which the person is located, each voxel having a respective numerical value that represents a magnitude and a phase of the reflected FMCW wireless signal(s) that was/were reflected from the respective physical location, and providing the three-dimensional reflected wireless signal map as the input to the trained ML model, the trained ML model having been trained with ground-truth three-dimensional wireless signal maps with respect to time.

Another aspect of the invention is directed to a system for determining an inflammation state of a person under observation, comprising: a wireless-tracking system and a computer. The wireless-tracking system comprises one or more transmitting antennas configured to transmit frequency-modulated continuous-wave (FMCW) wireless signals; one or more receiving antennas configured to receive reflected FMCW wireless signals, at least some of the reflected FMCW wireless signals being reflected, partially or fully, from a person under observation; a first processor circuit electrically coupled to the one or more transmitting antennas and the one or more receiving antennas; a power supply electrically coupled to the first processor circuit; and a first non-transitory computer-readable memory in electrical communication with the first processor circuit, the first non-transitory computer-readable memory storing computer-readable instructions that, when executed by the first processor circuit, cause the first processor circuit to: produce reflected FMCW wireless data based on the reflected FMCW wireless signals; and send the reflected FMCW wireless data to a computer. The computer comprises: a second processor circuit; a second non-transitory computer-readable memory in electrical communication with the second processor circuit, the second non-transitory computer-readable memory storing computer-readable instructions that, when executed by the second processor circuit, cause the second processor circuit to: store the reflected FMCW wireless data in the second non-transitory computer-readable memory; provide the reflected FMCW wireless data as an input to a trained machine-learning (ML) model, the trained ML model having been trained with ground-truth inflammation that represents ground-truth inflammation states of one or more subjects with respect to time and with ground-truth reflected FMCW wireless data of the one or more subjects with respect to time; and predict, with the trained ML model, whether the person under observation is in an inflamed state or in a non-inflamed state.

In one or more embodiments, the computer-readable instructions stored on the first non-transitory computer-readable memory, when executed by the first processor circuit, cause the first processor circuit to: convert a plurality of discrete time periods of raw reflected FMCW wireless data into respective three-dimensional reflected wireless signal maps, each three-dimensional reflected wireless signal map including a plurality of voxels that correspond to a respective physical location in a room in which the person under observation is located, each voxel having a respective numerical value that represents a magnitude and a phase of the reflected FMCW wireless signal(s) that was/were reflected from the respective physical location; and send three-dimensional reflected wireless signal maps to the computer, and the computer-readable instructions stored on the second non-transitory computer-readable memory, when executed by the second processor circuit, cause the second processor circuit to: store the three-dimensional reflected wireless signal maps in the second non-transitory computer-readable memory; determine a health indicator of the person under observation based on a plurality of the three-dimensional reflected wireless signal maps; determine one or more quantifiable health metrics related to the health indicator; and provide the quantifiable health metric(s) as the input the trained ML model, the trained ML model having been trained with ground-truth inflammation that represents ground-truth quantifiable health metric data of the one or more subjects with respect to time.

### BRIEF DESCRIPTION OF THE DRAWINGS

Fora fuller understanding of the nature and advantages of the present concepts, reference is made to the detailed description of preferred embodiments and the accompanying drawings.

FIG. 1 is a block diagram of a motion-tracking system according to an embodiment.

FIG. 2A is a plot of transmit and receive frequencies with respect to time.

FIG. 2B is a plot of received energy with respect to frequency corresponding to FIG. 2A.

FIG. 3A is a spectrogram (spectral profile versus time) for one transmit and receive antenna pair.

FIG. 3B is the spectrogram of FIG. 3A after background subtraction.

FIG. 3C is a plot of estimates of a first round-trip time corresponding to the spectrogram in FIG. 3B.

FIG. 4 is a block diagram of a motion-tracking system and an optional computer according to an embodiment.

FIG. 5 is a block diagram of an example room in which embodiment the motion-tracking system of FIG. 4 is placed to monitor a subject.

FIG. 6 is a flow chart of a method for predicting the inflammation state of a person according to an embodiment.

FIG. 7A is a simplified two-dimensional representation of a reflected wireless data map according to an embodiment.

FIG. 7B illustrates an example heatmap of accumulated position data according to an embodiment.

FIG. 8 illustrates an example structure of a recurrent neural network according to an embodiment.

FIG. 9 illustrate an example structure of a convolutional neural network with a single hidden layer according to an embodiment.

FIG. 10 is a flow chart of a method for training a machine-learning model according to an embodiment.

FIG. 11 is a flow chart of a computer-implemented method for predicting the inflammatory state of a subject according to an embodiment.

FIGS. 12A and 12B are example graphs that illustrate a subject's average gait speed and average breathing rate, respectively, with respect to time according to an embodiment.

FIG. 12C is an example graph that illustrates a predicted inflammatory state of the subject according to an embodiment.

### DETAILED DESCRIPTION

A wireless motion tracking system is used to collect wireless reflection data that represents the movement of a person in an environment such as a room. The wireless reflection data and/or higher-level quantifiable metric data that is based on the wireless reflection data are provided as input(s) to a trained machine-learning model to predict the inflammation state of the person. The trained machine-learning model was trained using (a) ground-truth inflammation data that represents ground-truth inflammation states of one or more subjects with respect to time and (b) ground-truth wireless reflection data and/or ground-truth higher-level quantifiable metric data of one or more subjects with respect to time.

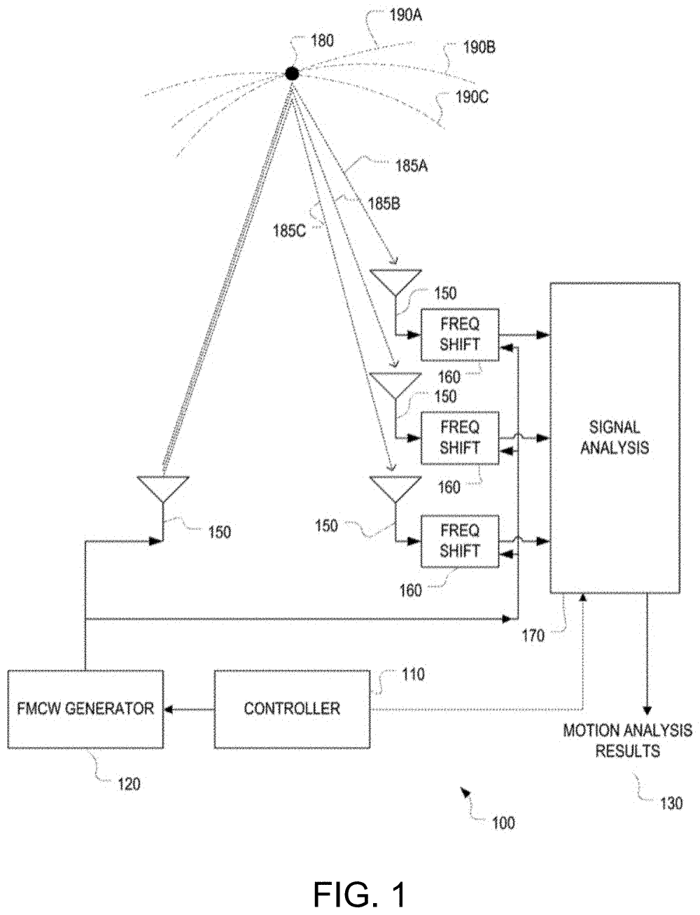

FIG. 1 is a block diagram of a motion-tracking system 100 according to an embodiment. The motion tracking system 100 includes multiple antennas 150 that transmit and receive radio-frequency (RF) signals that are reflected, partially or fully, from objects (e.g., people) in the environment of the system 100, which may include one or more rooms of a building, the interior of a vehicle, etc., and may be partitioned, for example, by substantially radio-transparent barriers, for instance building walls or cloth sheets. In general, the objects in the environment include both fixed objects, such as chairs, walls, etc., as well as moving objects, such as but not limited to people. The system 100 can track people, who may be moving around a room, getting out of a sleep platform (e.g., a bed), or may be relatively stationary, for instance, sitting in a chair or lying in bed, but may nevertheless exhibit breathing motion that may be detected by one or more embodiments described below. The system 100 provides motion analysis results 130 based on the radio frequency signals. In various embodiments, these results include locations of one or more people in the environment, detected body or limb gestures made by the people in the environment, detected physical activity including detection of falls, and/or the detected respiration of the people in the environment.

Generally, the system 100 makes use of time-of-flight (TOF) (also referred to as “round-trip time”) information derived for various pairs of antennas 150. For example, in schematic illustration in FIG. 1, three paths 185A-C reflecting off a representative point object 180 are shown between a transmitting antenna 150 and three receiving antennas 150, each path generally having a different TOF. Assuming a constant signal propagation speed c (i.e., the speed of light), the TOF from an antenna at coordinates (xₜ, yₜ, zₜ) reflecting from an object at coordinates (xₒ, yₒ, zₒ) and received at an antenna at coordinates (xᵣ, yᵣ, zᵣ) can be expressed as Equation 1:

$$\frac{1}{c} ( \sqrt{{( {x}_{t} - {x}_{o} )}^{2} + {( {y}_{t} - {y}_{o} )}^{2} + {( {z}_{t} - {z}_{o} )}^{2}} + \sqrt{{( {x}_{r} - {x}_{o} )}^{2} + {( {y}_{r} - {y}_{o} )}^{2} + {( {z}_{r} - {z}_{o} )}^{2}} ) . \qquad ( 1 )$$

For a particular path, the TOF, for example associated with path 185A, constrains the location of the object 180 to lie on an ellipsoid defined by the three-dimensional coordinates of the transmitting and receiving antennas of the path, and the path distance determined from the TOF. For illustration, a portion of the ellipsoid is depicted as the elliptical line 190A. Similarly, the ellipsoids associated with paths 185B-C are depicted as the lines 190B-C. The object 180 lies at the intersection of the three ellipsoids.

Continuing to refer to FIG. 1, the system 100 includes a signal generator that generates repetitions of a signal pattern that is emitted from the transmitting antenna 150. In this embodiment, the signal generator is an ultra-wide band frequency-modulated continuous-wave (FMCW) generator 120. It should be understood that in other embodiments other signal patterns and bandwidth(s) than those described herein may be used while following other aspects of the described embodiments. The FMCW signals can function as a type of radar that can track relative movement of objections that reflect the FMCW signals.

Additional details regarding the motion tracking systems, methods, and/or other aspects of this disclosure are described in the following documents, which are hereby incorporated by reference: U.S. Pat. No. 10,746,852, titled “Vital Signs Monitoring Via Radio Reflections,” which issued on Oct. 18, 2020; U.S. Pat. No. 9,753,131, titled “Motion Tracking Via Body Radio Reflections,” which issued on Sep. 5, 2017; U.S. Patent Application Publication No. 2020/0341115, titled “Subject identification in behavioral sensing systems,” which published on Oct. 29, 2020; U.S. Patent Application Publication No. 2019/0188533, titled “Pose Estimation,” which published on Jun. 20, 2019; U.S. Patent Application Publication No. 2018/0271435, titled “Learning Sleep Stages From Radio Signals,” which published on Sep. 27, 2018; and/or U.S. Patent Application Publication No. 2017/0311901, titled “Extraction Of Features From Physiological Signals,” which published on Nov. 2, 2017.

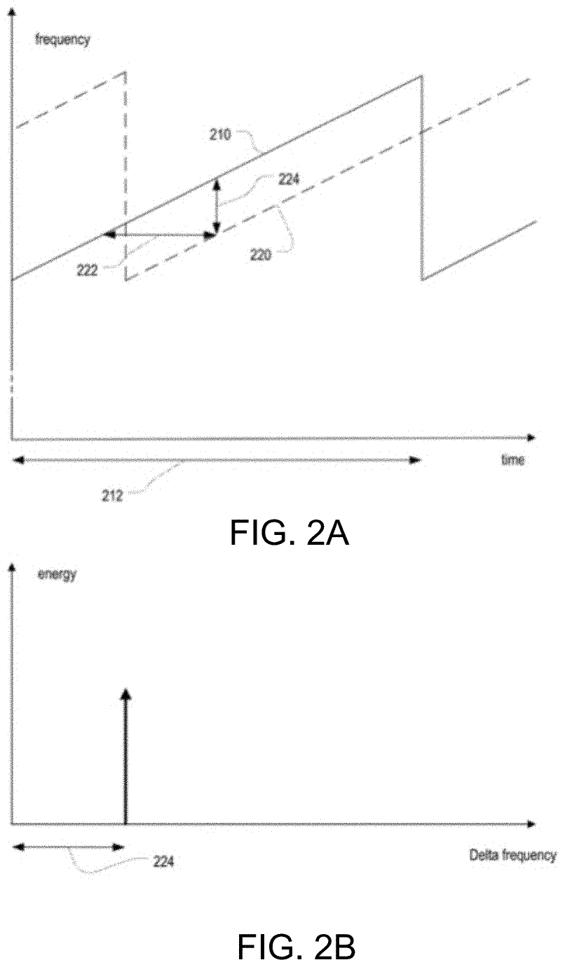

Referring to FIG. 2A, TOF estimates are made using a FMCW approach. Considering a single transmit and receive antenna pair, in each of a series of repeating time intervals 212 of duration T, a transmit frequency is swept over a frequency range as shown by solid line 210. In some embodiments, the frequency range is about 5.46-7.25 GHz (i.e., a frequency range of about 1.8 GHz) with a sweep duration and repetition rate of about 2.5 milliseconds. The receiving antenna receives the signal after a TOF 222 (i.e., reflected from a single object), with frequency as shown in the dashed line 220. Note that the TOF 222 corresponds to a difference 224 in transmitted and received frequencies, which is a product of the TOF and the rate of frequency change of the swept carrier for the transmit antenna.

Referring to FIG. 2B, if the received reflected signal (dashed line 220 in FIG. 2A) is frequency-shifted according to the transmitted signal (solid line 210 in FIG. 2A), then the result will have energy concentrated at the frequency difference 224 corresponding to the TOF. Note that we are ignoring the edges of the intervals 212, which are exaggerated in the figures. Referring back to FIG. 1, a frequency shift component 160 (also referred to as a “downconverter” or a “mixer”) implements the frequency shifting, for example, including a modulator that modulates the received signal with the transmitted signal to retain a low frequency range representing TOF durations that are consistent with the physical dimensions of the environment.

The output of the frequency shifter 160 is subject to a spectral analysis 170 (e.g., a Fourier transform), for example in a spectral analysis processor circuit, to separate the frequency components each associated with a different TOF. In this embodiment, the output of the frequency shifter is sampled and a discrete time Fourier transform, implemented as a fast Fourier transform (FFT), is computed for each interval 212. Each complex value of the FFT provides a frequency component with a frequency resolution

$$Δ f = \frac{n}{{T}_{s w e e p}}$$

where T_{sweep} is the sweep duration (e.g., 2.5 milliseconds) and n is the number of samples per sweep.

Continuing to refer to FIG. 2B, it should be recognized that the distribution of energy over frequency (and equivalently over TOF), is not generally concentrated as shown in this figure. Rather, there is a distribution of energy resulting from the superposition of reflections from the reflective objects in the environment. Some reflections are direct, with the path being direct between the reflecting object and the transmitting and receiving antennas. Other reflections exhibit multipath effects in which there are multiple paths from a transmitting antenna to a receiving antenna via a particular reflecting object. Some multipath effects are due to the transmitted signal being reflected off walls, furniture, and other static objects in the environment. Other multipath effects involve reflection from a moving body, where the path is not direct from the transmitting antenna to a moving object and back to the receiving antenna, but rather is reflected from one or more static objects either on the path from the transmitting antenna to the moving object or from the moving object back to the receiving antenna, or both.

The system 100 addresses the first multipath effect, referred to as static multipath, using a time-differencing approach to distinguish a moving object's reflections from reflections off static objects in the environment, like furniture and walls. Typically, reflections from walls and furniture are much stronger than reflections from a human, especially if the human is behind a wall. Unless these reflections are removed, they would mask the signal coming from the human and prevent sensing her motion. This behavior is called the “Flash Effect”.

To remove reflections from all of these static objects (e.g., walls, furniture), we leverage the fact that since these reflectors are static, their distance to the antenna array does not change over time, and therefore their induced frequency shift stays constant over time. We take the FFT of the received signal every sweep window and eliminate the power from these static reflectors by subtracting the (complex) output of the FFT in a given sweep from the FFT of the signal in a previous sweep. This process is called background subtraction because it eliminates all the static reflectors in the background. In some embodiments, the immediately-previous sweep (i.e., 2.5 milliseconds previous) is background subtracted, while in other embodiments, a greater delay may be used (e.g., 12.5 milliseconds, or even over a second, such as 2.5 seconds) to perform background subtraction.

By eliminating all reflections from static objects, the system is ideally left only with reflections from moving objects. However, as discussed above, these reflections include both signals that travel directly from the transmitting antenna to the moving body (without bouncing off a static object), reflect off the object, and then travel directly back to the receiving antenna, as well as indirect paths that involve reflection from a static object as well as from a moving object. We refer to these indirect reflections as dynamic multi-path. It is quite possible that a moving object reflection that arrives along an indirect path, bouncing off a side wall, is stronger than a direct reflection (which could be severely attenuated after traversing a wall) because the former might be able to avoid occlusion.

The general approach to eliminating dynamic multi-path is based on the observation that, at any point in time, the direct signal paths to and from the moving object have traveled a shorter path than indirect reflections. Because distance is directly related to TOF, and hence to frequency, this means that the direct signal reflected from the moving object would result in the smallest frequency shift among all strong reflectors after background subtraction. We can track the reflection that traveled the shortest path by tracing the lowest frequency (i.e., shortest time of flight) contour of all strong reflectors.

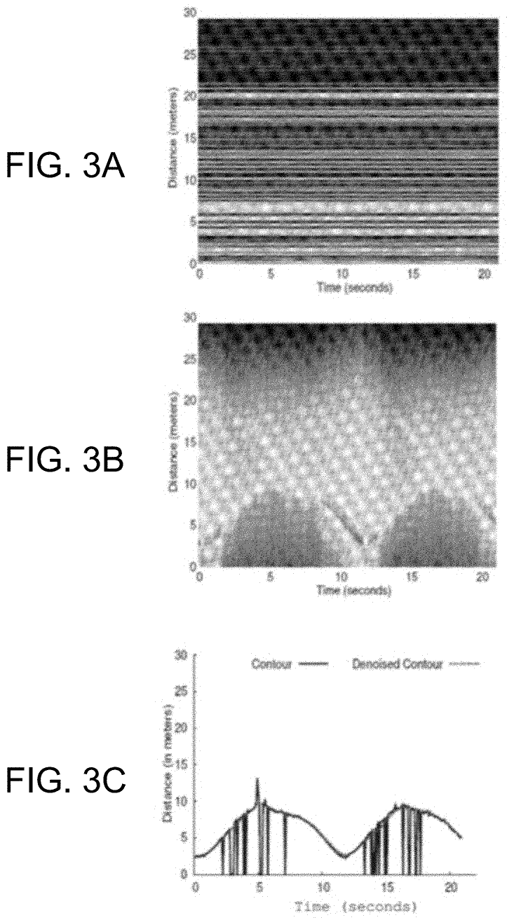

Referring to FIGS. 3A-C, the horizontal axis of each figure represents a time interval of approximately 20 seconds, and the vertical axis represents a frequency range corresponding to zero distance/delay at the bottom to a frequency corresponding to a range of approximately 30 meters at the top. FIGS. 3A and 3B show FFT power before background subtraction (FIG. 3A) and after background subtractions (FIG. 3B).

FIG. 3C shows the successive estimates of the shortest distance (or equivalently time) of flight for successive sweeps, as well as a “denoised” (e.g., smoothed, outlier eliminated, etc.) contour.

To determine the first local maximum that is caused by a moving body, we must be able to distinguish it from a local maximum due to a noise peak. We achieve this distinguishability by averaging the spectrogram across multiple sweeps. In this embodiment, we average over five consecutive sweeps, which together span a duration of 12.5 milliseconds, prior to locating the first local maximum of the FFT power. For all practical purposes, a human can be considered as static over this time duration; therefore, the spectrogram (i.e., spectral distribution over time) would be consistent over this duration. Averaging allows us to boost the power of a reflection from a moving body while diluting the peaks that are due to noise. This is because the human reflections are consistent and hence add up coherently, whereas the noise is random and hence adds up incoherently. After averaging, we can determine the first local maximum that is substantially above the noise floor and declare it to be the direct path to the moving body (e.g., a moving human).

In practice, this approach using the first reflection time rather than the strongest reflection proves to be more robust, because, unlike the contour which tracks the closest path between a moving body and the antennas, the point of maximum reflection may abruptly shift due to different indirect paths in the environment or even randomness in the movement of different parts of a human body as a person performs different activities.

Note that the process of tracking the contour of the shortest time of flight is carried out for each of the transmitting and receiving antenna pairs, in this embodiment, for the three pairs each between the common transmitting antenna and the three separate receiving antennas. After obtaining the contour of the shortest round-trip time for each receive antenna, the system leverages common knowledge about human motion to mitigate the effect of noise and improve its tracking accuracy. The techniques used include outlier rejection, interpolation, and/or filtering.

In outlier rejection, the system rejects impractical jumps in distance estimates that correspond to unnatural human motion over a very short period of time. For example, in FIG. 3C, the distance from the object repeatedly jumps by more than 5 meters over a span of few milliseconds. Such changes in distance are not possible over such small intervals of time, and hence the system rejects such outliers.

In interpolation, the system uses its tracking history to localize a person when she stops moving. In particular, if a person walks around in a room, then sits on a chair and remains static, the background-subtracted signal would not register any strong reflector. In such scenarios, we assume that the person is still in the same position and interpolate the latest location estimate throughout the period during which we do not observe any motion, enabling us to track the location of a subject even after she stops moving.

In filtering, because human motion is continuous, the variation in an object's distance to each receive antenna should stay smooth over time. Thus, the system uses a filter, such as a Kalman filter, to smooth the distance estimates.

After contour tracking and de-noising of the estimate, the system obtains a clean estimate of the distance traveled by the signal from the transmit antenna to the moving object, and back to one of the receive antennas (i.e., the round-trip distance). In this embodiment that uses one transmitting antenna and three receiving antennas, at any time, there are three such round-trip distances that correspond to the three receive antennas. The system uses these three estimates to identify the three-dimensional position of the moving object, for each time instance.

The system leverages its knowledge of the placement of the antennas. In this embodiment, the antennas are placed in a “T” shape, where the transmitting antenna is placed at the cross-point of the “T” and the receiving antennas are placed at the edges, with a distance of about 1 meter between the transmitting antenna and each of the receiving antennas. For reference, the z axis refers to the vertical axis, the x axis is along the horizontal, and, with the “T” shaped antenna array mounted to a wall, the y axis extends into the room. Localization in three dimensions uses the intersection of the three ellipsoids, each defined by the known locations of the transmitting antenna and one of the receiving antennas, and the round-trip distance.

Note that in alternative embodiments, only two receiving antennas may be used, for example with all the antennas placed along a horizontal line. In such an embodiment, a two-dimensional location may be determined using an intersection of ellipses rather than an intersection of ellipsoids. In other alternative embodiments, more than three receiving antennas (i.e., more than three transmitting-receiving antenna pairs) may be used. Although more than three ellipsoids do not necessarily intersect at a point, various approaches may be used to combine the ellipsoids, for example, based on a point that is closest to all of them.

For example, the antennas can include an antenna array in which antenna elements are distributed in two dimensions. A particularly useful embodiment is an antenna array in which antenna elements are arranged at regular intervals along vertical and horizontal axes. This arrangement can provide a particularly convenient way to separate signals that come from different locations in the three-dimensional space.

A frame generator can process the data that arrives from the receiving antennas to form RF frames or “frames.” The frame generator can use the antenna elements along the horizontal axis to generate successive two-dimensional horizontal frames and can use the antenna elements along the vertical axis to generate successive two-dimensional vertical frames. The horizontal frames are defined by a distance axis and a horizontal-axis. The vertical frames are defined by the same distance axis and a vertical-angle axis. The horizontal and vertical frames can therefore be viewed as projections of the frame into two subspaces.

The frame generator can generate data indicative of a reflection from a particular distance and angle. It does so by evaluating a double-summation for the horizontal frames and another double-summation for the vertical frames.

The double-summations are identical in form and differ only in details related to the structures of the horizontal and vertical lines of antenna elements and in whether the vertical or horizontal angle is being used. Thus, it is sufficient to show only one of the double summations below in Equation 2:

$$P ( d , θ ) = {\sum }_{n = 1}^{N} {\sum }_{t = 1}^{T} {s}_{n , t} {e}^{j 2 π kdt / c} {e}^{j 2 π nl cos ( θ ) / λ} \qquad ( 2 )$$

In Equation 2, P(d, θ) represents the value of the reflection from a distance d in the direction θ, s_{n,t} represents the t^{th} sample of the reflected chirp (e.g., transmitted signal pattern) as detected by the nth antenna element in the line of antenna elements, c and A represent the radio wave's velocity and its relevant wavelength, respectively, N represents the total number of antenna elements in the relevant axis of antenna elements, T represents the number of samples from the relevant reflection of the outgoing chirp, l represents the spatial separation between adjacent antenna elements, and k represents the slope of the chirp in the frequency domain.

As a result, at each step, it is possible to represent the reflected signal using its projection on a horizontal plane and on a vertical plane. The horizontal frame captures information concerning a subject's location and the vertical frame captures information concerning the subject's build, including such features as the subject's height and girth. Differences between successive frames in a sequence of horizontal and vertical frames can provide information concerning each subject's characteristic gait and manner of movement. A preferred embodiment operates at a frame rate of thirty frames-per-second. This is sufficient to assume continuity of locations. Additional details regarding the antenna arrangement and frame calculation are disclosed in U.S. Patent Application Publication No. 2020/0341115.

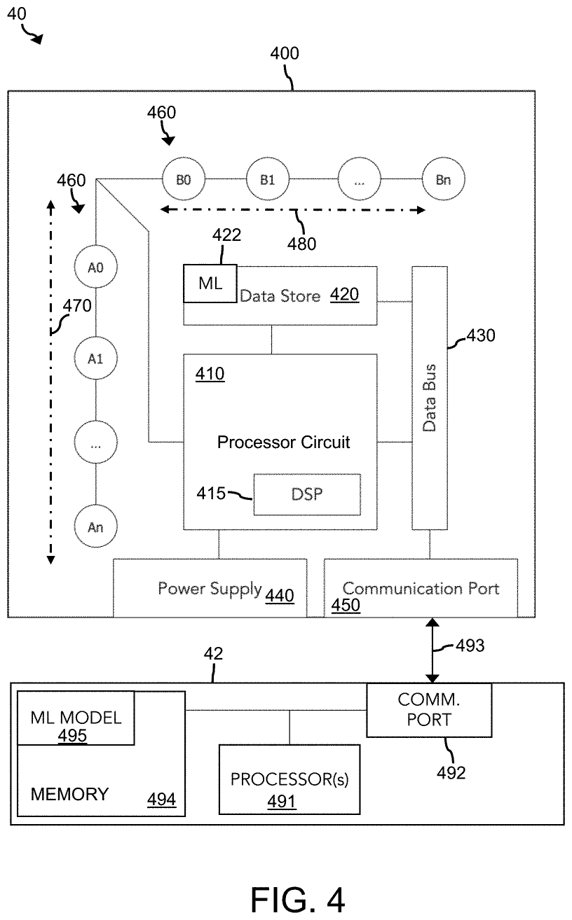

FIG. 4 is a block diagram of a motion tracking system 40 according to another embodiment. The motion tracking system 40 can be the same as motion tracking system 100. The motion tracking system 40 includes a housing 400, a processor circuit 410, a data store 420, a data bus 430, a power supply 440, an optional communication port 450, and a plurality of RF antennas 460. The housing 400 can contain some or all components of the system 40 including the processor circuit 410, the data store 420, the data bus 430, the power supply 440, the optional communication port 450, and/or the RF antennas 460. In some embodiments, the RF antennas 460 are positioned and/or distributed along one or more walls in a room. The antennas 460 can be located in the same housing or in a different housing as the processor circuit 410, the data store 420, the data bus 430, and/or the power supply 440. Alternatively, the antennas 460 can be mounted on a wall or other surface and may not be located in a housing. The housing 400 can comprise plastic, ceramic, or another material. The housing 400 is preferably at least partially transparent to the RF frequency(ies) emitted and/or received by the RF antennas 460. For example, the housing 400 can provide minimal (e.g., less than 5-10%) or no attenuation of the RF signals emitted and/or received by the RF antennas 460. In some embodiments, some or all of the components can be mounted on or electrically connected to a printed circuit board. In some embodiments, the RF antennas 460 can be replaced by other energy-emission devices such as ultrasonic transducers.

The processor circuit 410 can comprise an integrated circuit (IC) such as a microprocessor, an application-specific IC (ASIC), or another hardware-based processor. For example, the microprocessor can comprise a central processing unit (CPU), a graphics processing unit (GPU), and/or another processor. The processor circuit 410 also includes one or more digital signal processors (DSPs) 415 that can drive the RF antennas 460. The construction and arrangement of the processor circuit 410 can be determined by the specific application and availability or practical needs of a given design. The processor circuit 410 can include the controller 110, the FMCW generator 120, and/or the frequency shift components 160 in motion-tracking system 100.

The processor circuit 410 is electrically coupled to the power supply 440 to receive power at a predetermined voltage and form. For example, the power supply 440 can provide AC power, such as from household AC power, or DC power such as from a battery. The power supply 440 can also include an inverter or a rectifier to convert the power form as necessary (e.g., from AC power to DC power or from DC power to AC power, respectively).

The optional communication port 450 can be used to send and/or receive information or data to and/or from a second device, such as a computer, a sensor (e.g., in the subject's bedroom), a server, and/or another device. The optional communication port 450 can provide a wired or wireless connection for communication with the second device. The wireless connection can comprise a LAN, WAN, WiFi, cellular, Bluetooth, or other wireless connection. In some embodiments, the optional communication port 450 can be used to communicate with multiple devices.

The data store 420 can include non-transitory computer-readable memory (e.g., volatile and/or non-volatile memory) that can store data representing stored machine-readable instructions and/or data collected by the system 40 or intermediate data used by the processor circuit 410. The data bus 430 can provide a data connection between the data store 420, the processor circuit 410, and/or the optional communication port 450. The data store 420 can also include program instructions that represent artificial intelligence, a trained neural network (e.g., a convolutional neural network), and/or machine learning that can be trained to perform one or more tasks of the technology. The program instructions are executable by the processor circuit 410.

The RF antennas 460 can include or consist of one or more dedicated transmitting antennas and/or one or more dedicated receiving antennas. Additionally or alternatively, one or more of the RF antennas 460 can be a transceiver antenna (e.g., that can transmit and receive signals but preferably not simultaneously). Any receiving antenna 460 can receive a signal from any transmitting antenna 460. In one example, an antenna sweep can occur by sending a first signal from a first transmitting antenna 460, which is received by one or more receiving antenna(s) 460. Next, a second transmitting antenna 460 can send a second signal, which can be received by one or more receiving antenna(s) 460. This sweep process can continue where each transmitting antenna 460 sequentially sends a respective signal that is received by one or more receiving antenna(s) 460. Each signal can be reflected by the subject partially or fully. The RF antennas 460 are electrically coupled to the processor circuit 410 and the power supply 440.

The RF antennas 460 can be arranged with respect to one another to form one or more wireless transmit-receive arrays. As illustrated, a first group or array of RF antennas 460 (A0, A1 . . . An) is placed along or parallel to a first axis 470 and a second group or array of RF antennas 460 (B0, B1 . . . Bn) is placed along or parallel to a second axis 480 that is orthogonal to the first axis 470, for example in an “L” arrangement. The first and second groups of RF antennas 460 can be configured and arranged to capture a spatial position of the target of interest, e.g., the subject's body and the bed. It is noted that the first and second groups of RF antennas 460 can be arranged in different relative orientations to achieve the same or substantially the same result. For example, the first and second axes 460, 470 can disposed at other angles with respect to each other that are different than 90°. Alternatively, the first and second groups of RF antennas 460 can be configured in a “T” arrangement or a “+” instead of the “L” arrangement illustrated in FIG. 4.

In addition or in the alternative, one group of RF antennas 460 can be used to collect reflection and position data in the (x, y) plane, which can be defined as parallel to the floor or sleeping surface planes, and the other group of RF antennas 460 can be used to optionally collect height or elevation data regarding the third (z) axis position. In another embodiment, each group of RF antennas 460 can include one or more dedicated transmit antennas, one or more dedicated receive antennas, and/or one or more dedicated transmit antennas and one or more dedicated receive antennas. The RF antennas 460 in each group can be spaced at equal distances from each other, or they may not be, and may even be randomly spaced or distributed in the housing 400. Alternatively, those skilled in the art will understand that the system may be constructed using more than one physical device that can be distributed spatially about an indoor environment such as a bedroom, hospital room or other space, i.e., placing separate components within multiple distinct housings.

In a preferred but not limiting example, the RF antennas 460 can span a finite spatial extent which allows for localization or triangulation to determine the position of a reflecting body, e.g., the subject's body. For example, the RF antennas 460 can emit RF signals with a wavelength in a range of about 0.01 meter (i.e., about 1 cm) to about 1 meter, including any value or range therebetween. In another example, the RF antennas 460 can emit RF signals with a wavelength in a range of about 1 cm to about 10 cm, including any value or range therebetween. Electromagnetic radiation waves with wavelengths greater than 1 mm are preferred, i.e., those wavelengths substantially greater than the wavelengths of visible light.

The motion tracking system 40 can be programmed, configured, and/or arranged to perform motion tracking automatically, for example using the RF antennas 460 and processing components. Therefore, the motion tracking system 40 can continuously and inexpensively monitor a human subject in an indoor environment such as a bedroom, though sometimes the present system can emit and receive wireless signals through walls, furniture or other obstructions to extend its versatility and range. Also, the motion tracking system 40 can continuously monitor, detect, and/or and measure the data required to determine or estimate one or more health indicators of a subject under observation. Furthermore, the motion tracking system 40 can be deployed to an observation space (e.g., a subject's bedroom) in a non-obtrusive form factor such as a bedside apparatus or an apparatus inconspicuously mounted on a wall, ceiling, or fixture of the subject's room.

The motion tracking system 40 can be coupled to a communication network (e.g., using optional communication port 450) so that data collected may be analyzed remotely, off-line, or in real time by a human or a machine (e.g., computer, server, etc.) coupled to the network. For example, the optional communication port 450 can be used to deliver data over the network (e.g., the internet) to a server, clinical station, medical care provider, family member, or other interested party. Data collected can be archived locally in memory (e.g., data store 420) of the system 40 or may be transmitted (e.g., using optional communication port 450) to a data collection or storage unit or facility over a wired or wireless communication channel or network.

A processor-implemented or processor-assisted method can be carried out automatically or semi-automatically. The processing may take place entirely on internal processing components in the system 40 and/or at a remote processing location. For example, the system 40 can receive the reflected wireless data (e.g., raw reflected wireless data) and send that data to be processed remotely such as at a cloud-based server. In yet other embodiments a hybrid arrangement may be used where processing is performed at both at the local device (e.g., in system 40) and remotely. Therefore, for the present purposes, unless described otherwise, the location of the processing acts is not material to most or all embodiments.

In an embodiment, the motion-tracking system 40 can be in electrical communication with a computer 42. The computer 42 includes one or more processor circuits 491, which can be the same as or different than processor circuit 410. The processor circuit(s) 491 is/are in electrical communication with a communication port 492 that can provide a wired or wireless data connection 493 with the motion tracking system 40 (e.g., via the communication port 450 of the motion tracking system 40). The computer 42 can receive reflected wireless data (e.g., raw reflected wireless data and/or three-dimensional reflected wireless signal maps) and/or higher-level data (e.g., quantifiable health metric(s) as described herein) from the motion tracking system 40 via the wired/wireless connection 493. The reflected wireless data and/or higher-level data can be stored in computer memory 494.

The processor circuit(s) 491 is/are in electrical communication with computer memory 494. The computer memory 494 stores processor-readable or computer-readable instructions that are configured to be executed by the processor circuit(s) 491. The computer memory 494 can include or can be non-transitory computer memory. The computer memory 494 can store a trained machine-learning model 495 that can predict the inflammation state of the person (or more generally, mammal) under observation. The processor circuit(s) 491 can provide as input(s) to the trained machine-learning model 495 the reflected wireless data and/or higher-level data (e.g., quantifiable health metric(s) as described herein) that are received from the motion-tracking system 40. Additional details regarding the trained machine-learning model 493 are described herein.

In some embodiments, the motion-tracking system 40 only provides the reflected wireless data to the computer 42, in which case the computer 42 can be configured to analyze the reflected wireless data to produce the higher-level data (e.g., quantifiable health metric(s), which can be stored in the computer memory 494. In other embodiments, the higher-level data is not used as an input to the trained ML model in which case it is optional for the motion-tracking system 40 and/or the computer 42 to produce the higher-level data.

Additionally or alternatively, a trained ML model 422 can be stored in the data store 420 and executed by the processor circuit 410 in the motion-tracking system 40. The trained ML model 422 can be the same as or different than the trained ML model 495.

Additionally or alternatively, the computer 42 can compare the quantifiable health metric(s) with baseline quantifiable health metric(s) of the person, the person's age group, the person's gender, etc. to determine whether a flare-up (e.g., inflammation) has occurred.

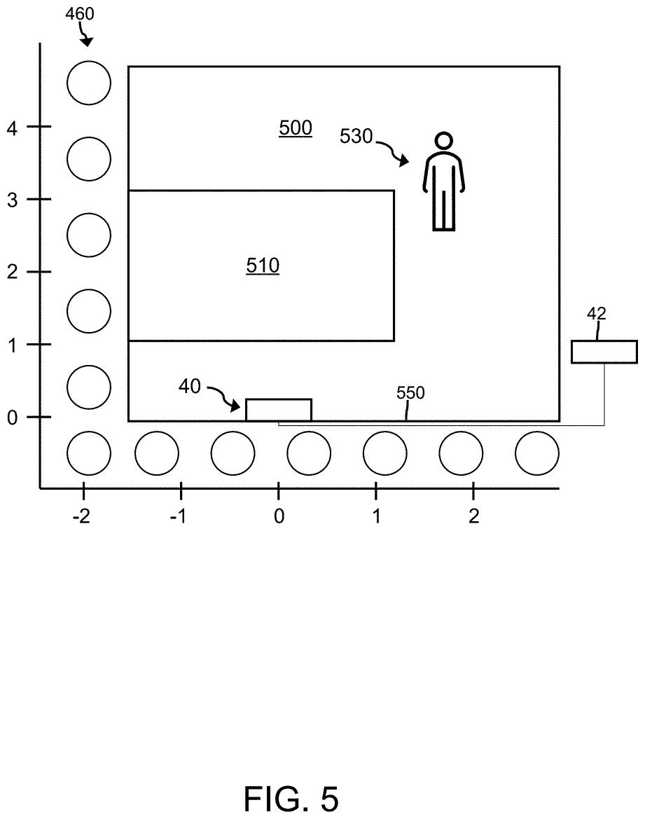

FIG. 5 is a block diagram of an example room 500 or other indoor environment in which embodiments of the technology can be applied. A typical home or institution or hospital room may be the general space within which most or all of the present steps are carried out. The room 500 includes a bed 510 or other sleep platform and the wireless motion-tracking system 40 located at a (0, 0) reference location, where the numbers along the two axes are indicated in meters. The area and position of the walls of the room 500 and its contents, including the bed 510 and/or other furnishings, can be automatically measured by the motion-tracking system 40, or can be entered manually, such as at the time of installation of the motion-tracking system 40, to identify or key in the relative dimensions or locations of key objects such as walls, beds, and so on.

The antennas 460 of the motion-tracking system 40 can be placed or mounted along or parallel to one or more walls 550 of the room 500. For example, the antennas 540 can be placed or mounted along or parallel to adjacent walls 550 that are oriented orthogonally with respect to each other, such as in the configuration of the antennas 460 illustrated in FIG. 4. The antennas 460 are preferably evenly spaced with respect to the respective walls 550.

The motion-tracking system 40 can be in electric communication with computer 42, such as in the embodiment illustrated in FIG. 4. The motion-tracking system 40 and/or the computer 42 includes a trained ML model (e.g., as discussed with respect to FIG. 4) that is configured to predict the inflammation state of a person 530 located in the room 500. The trained model can use as inputs the reflected wireless data and/or higher-level data (e.g., quantifiable health metric(s)) that can be determined using the reflected wireless data. Examples of quantifiable health metric(s) include the breathing rate (e.g., respiration rate), physical position, activity state (e.g., asleep or awake), sleep stage, gate speed, and/or other quantifiable health metric(s) of the person 530.

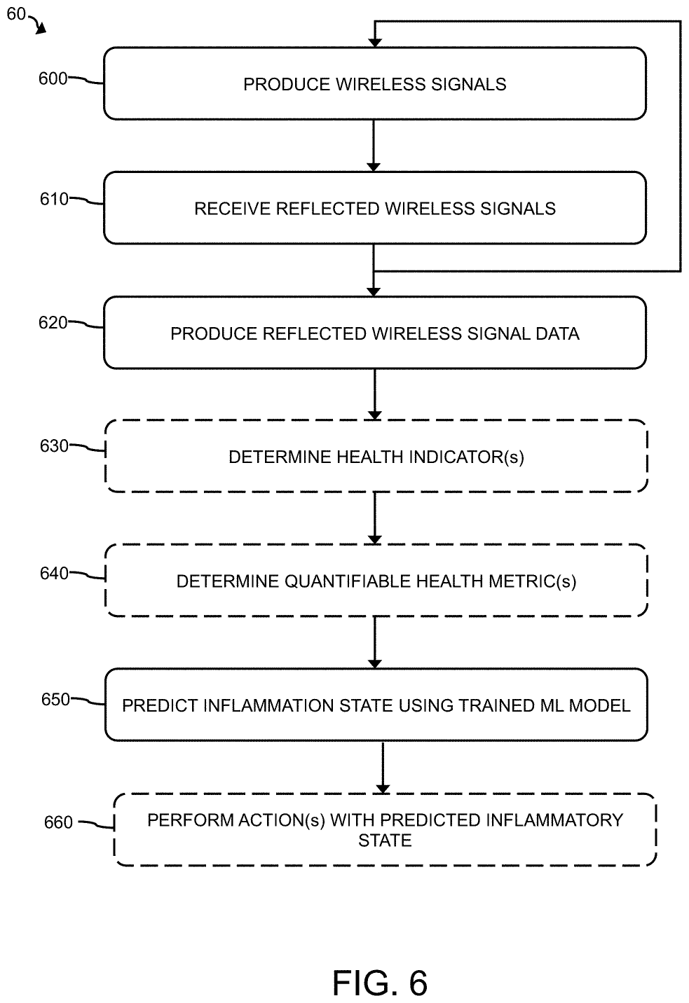

FIG. 6 is a flow chart of a method 60 for predicting the inflammation state of a person according to an embodiment. Method 60 can be implemented using motion-tracking system(s) 100 and/or 40 and/or using motion-tracking system 40 and computer 42.

In step 600, FMCW wireless signals are produced by one or more transmitting antennas. The transmitting antenna(s) can be the same as antennas 150, 460. At least some of the wireless signals are transmitted towards a person, such as towards the person 530 in room 500.

In step 610, reflected FMCW wireless signals are received by one or more receiving antennas. The reflected wireless signals can be reflected by the person 530, walls 550 in the room 500, and/or furniture (e.g., bed 510) in the room 500. The receiving antennas can be the same as antennas 150, 460. The receiving antenna(s) can be the same as or different than the transmitting antenna(s). In addition, the number and/or arrangement of the receiving antenna(s) can be the same as or different than the number and/or arrangement of the transmitting antenna(s). The reflected FMCW wireless signals (e.g., FMCW data) can function as a type of radar.

Steps 600 and 610 can be repeated while the person is under observation. For example, steps 600 can 610 can be repeated continuously, such as multiple times per second (e.g. 5-10 times per second), while the person is under observation. The method 60 can proceed to step 620 while steps 600 and 610 are repeated.

In step 620, the motion-tracking system produces reflected wireless data based on or using the reflected FMCW wireless signals. In some embodiments, the reflected wireless data is or includes a raw digital representation of the reflected FMCW wireless signals (e.g., raw reflected FMCW wireless data). The digital representation can include the magnitude and phase of each reflected FMCW wireless signal, the time at which each reflected FMCW wireless signal is received by a receiving antenna, and optionally the identity of the receiving antenna.

Additionally or alternatively, the motion-tracking system converts a discrete time period (e.g., a snapshot) of raw reflected FMCW wireless data into a reflected wireless data map of the room in which the person under observation is located. The snapshot can correspond or be equal to the frequency at which steps 600 and 610 are repeated (e.g., multiple times per second such as 5-10 times per second). The reflected wireless data map can include a three-dimensional map can be discretized into individual voxels, with each voxel corresponding to a respective physical location in the room. A respective numerical value, in real or complex form, of each voxel represents the magnitude and phase of the wireless signal(s) reflected from the respective/corresponding physical location (e.g., point objects 180 (FIG. 1)). As steps 600 can 610 are repeated, multiple reflected wireless data maps can be produced.

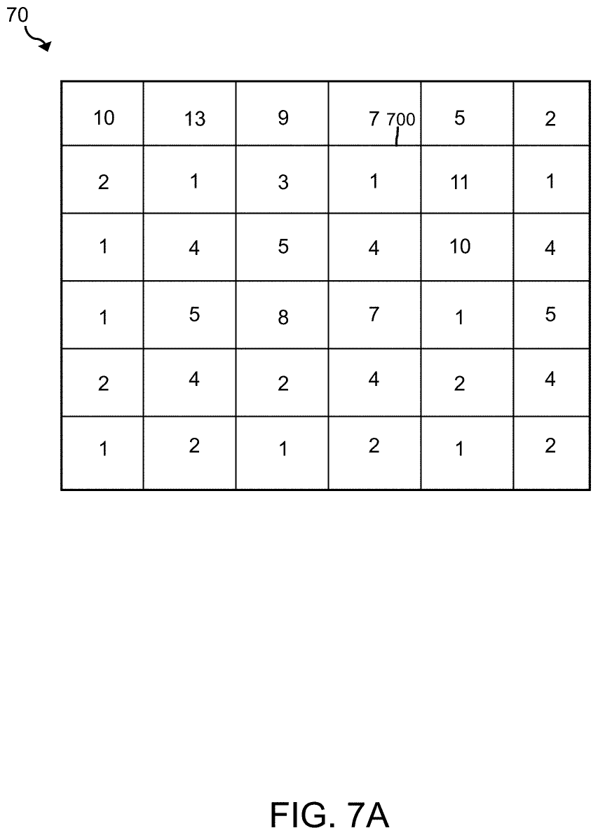

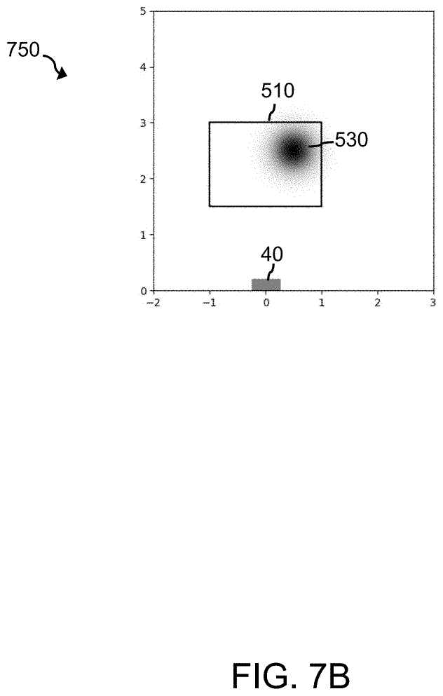

A simplified two-dimensional representation of a reflected wireless data map 70 is illustrated in FIG. 7A. The reflected wireless data map 70 includes a simplified two-dimensional representation of an array of voxels 700 where each voxel 700 has a respective numerical value.

In some embodiments, multiple reflected wireless data maps can be overlaid to form a heatmap. FIG. 7B illustrates an example heatmap 750 of accumulated position data (illustrated as dark color) indicating that the person 530 is located in the bed 510 for the majority of the observation time. The wireless motion-tracking system 40 can therefore observe and locate the subject during his or her sleep. In other embodiments, the heatmap can indicate that the person 530 is out of bed 510 and moving in the room 500.

Returning to method 60, in optional step 630, one or more health indicators of a person is/are determined using the reflected wireless signals. The health indicators can include the breathing rate (e.g., respiration rate), physical position, activity state (e.g., asleep or awake, laying down or standing), sleep stage, and/or other health indicia of the person 530.

Conclusions can be drawn regarding the person's sleep pattern and health from the aggregated position data, for example indicating how much the subject moves around in the sleep or laying-down area during a night's sleep or from night to night, and/or the sleep stages of the person. Conclusions can also be drawn regarding the person's respiration rate, movement within the room 500, and/or other motions.

A person's breathing signal is a time-series signal reflecting the intake of air into the lungs. As an example, the signal might indicate the volume of air in the lungs at each instant in time. A breathing signal can be extracted from a heatmap by observing changes in the voxel values from snapshot to snapshot. In particular, as a person's chest and abdomen are displaced by the motion inherent in breathing, the magnitude and/or phase of the radio waves reflected from the corresponding voxels changes. For example, if a person is breathing at a rate of 20 breaths per minute, the phase of the radio signal reflecting from the person's chest might oscillate with a period of 3 seconds. This oscillation in phase can be interpreted as the person's breathing signal.

In optional step 640, one or more quantifiable health metric(s) is/are determined. The quantifiable health metrics are related to and/or based on the health indicators determined in optional step 630.

In an embodiment, the breathing rate of the person can be determined using the breathing signal. For example, we can take a Fourier transform of the breathing signal for each 30-second period of observation. The frequency with the maximum peak value can be considered the average breathing rate of the person for that period. This way we can produce an average breathing rate for every 30-second period for which we have extracted a breathing signal. We can also average all the breathing rates measurements from one night of sleeping to produce the average breathing rate for the night.

Additionally or alternatively, the sleep stages of the person can be determined, for example as disclosed in U.S. Patent Application Publication No. 2018/0271435, incorporated by reference above. The sleep stages can be used to extract quantifiable health metrics, such as time spent in REM (rapid eye movement) sleep per day, time spent in deep sleep (e.g., slow-wave sleep) per day, average breathing rate during REM sleep, average breathing rate during deep sleep, etc.

Additionally or alternatively, the gait speed of the person can be determined. For example, the position of the person can be determined every 0.5 seconds or another frequency and the gait speed can be determined as the change in position with respect to time. An average, median, and/or maximum gait speed can be determined.

In some embodiments, one or more features of the quantifiable health metrics can be determined. For example, the variation (if any) in breathing rate over a given time period, the variation (if any) in breathing rate over a given time period, the length or percentage of each sleep stage (e.g., REM sleep, deep sleep), and/or other features.

In step 650, the inflammation state of the person is predicted using a trained ML model (e.g., trained ML model 422 and/or 495). The trained ML model can predict the inflammation state using the reflected wireless data and/or the quantifiable health metric(s). The reflected wireless data can include one or more (e.g., a plurality of) heatmaps (e.g., heatmap 750) that includes an array of voxels with each voxel corresponding to a respective physical location in the room in which the person under observation is located. The numerical value, in real or complex form, of each voxel represents the magnitude and phase of the wireless signal reflected from the respective/corresponding physical location.

In optional step 660, the computer or motion-tracking system performs one or more actions with the predicted inflammatory state. The action(s) can include storing the predicted inflammatory state in memory operatively coupled to the computer. Additionally or alternatively, the action(s) can include displaying the predicted inflammatory state on a display screen operatively coupled to the computer. Additionally or alternatively, the action(s) can include producing an output signal when the predicted inflammatory state is the inflammatory state (i.e., that the subject is in the inflammatory state). The output signal can be sent to a device or an account controlled or owned by the subject. For example, the output signal can be sent to the subject's email address, to the subject's smartphone (e.g., as a text sent to the subject's phone number), to the subject's data records (e.g., that can be accessed by the subject and his/her physician). Step 660 can be optional in some embodiments.

To train and validate a ML model, one needs to collect a ground truth dataset. For example, the wireless motion-tracking system can be used to monitor people who are known to have Crohn's disease. At the same time, blood and/or stool samples can be collected on a regular basis from the monitored people. The blood and/or stool samples can be sent to a lab to measure the level of inflammation in the bodies of the people. One such test is the C-reactive protein blood test, which measures the general level of inflammation in the body. Another example is measuring fecal calprotectin (F.Cal), which is a substance that the body releases when there is inflammation in the intestines. F.Cal is the gold standard for measuring inflammation from Crohn's disease. For each lab measurement, we can compare the result to reference or baseline values to determine if the person has abnormal test results and if so, we will say that the person is flaring. Otherwise, we will say that the person is in remission at the date the test was taken. For example, for F.Cal we will say the person is flaring if the rest result is above 100 μg/mg, and we will say that the person is in remission otherwise.

In an embodiment, the motion-tracking system initially collects data over a limited period of time, e.g., over several days and/or for limited number of subjects, to train an ML model such as a support vector classifier, a support vector machine, a neural network, and/or other ML models.

Using the collected dataset and the measurements from the wireless sensor, an ML classifier can be built to predict whether a person is flaring up or not. The ML classifier can use the quantifiable health metric(s) and optionally the raw reflected RF signals as inputs. One option is to use a support vector classifier (SVC) or a support vector machine (SVM), which is trained using as input a set of features X (called training observations) and the corresponding set of known ground-truth labels Y. A ground-truth label of 1 indicates a positive observation (e.g., inflammation) and a ground-truth label of −1 indicates a negative observation. In our case, the input features can be the quantifiable health metric(s) and optionally the raw reflected RF signals extracted from the previous 30 days preceding the date of the ground-truth measurement.

As an example, the quantifiable health metrics used as inputs to the SVC can consist of the average or median gait speed and average or median breathing rate for each of the past thirty days. The SVC can be trained to accept these sixty data points as a single test observation X and then to classify the test observation as either positive or negative, i.e., whether inflammation is present.

Training an SVC amounts to finding a hyperplane that separates the positive training observations from the negative training observations. A hyperplane is characterized by a vector (β₁, . . . , βₚ) that is orthogonal to the hyperplane, and an offset β₀. A point (x₁, . . . , xₚ) is on the hyperplane if β₀+β₁xᵢ₁+β₂xᵢ₂+ . . . +βₚxᵢₚ=0. The hyperplane separates the positive and negative training observations so that for all positive observations (x₁, . . . , xₚ), β₀+β₁x₂+β₂x₂+ . . . +βₚxₚ>0, and for all negative observations (x₁, . . . , xₚ), β₀+β₁x₁+β₂x₂+ . . . +βₚxₚ<0. A new observation (x₁, . . . , xₚ) is classified by computing β₀+β₁x₁+β₂x₂+ . . . +βₚxₚ. If this quantity is positive, the observation is classified as positive, otherwise it is classified as negative.

There may be more than one hyperplane that separates the positive training observations from the negative training observations. Hence, to improve the accuracy of the classifier, the hyperplane is chosen that most separates the positive observations from the negative observations in the sense that there is the greatest distance, or “margin” M from any training observation to the hyperplane. This hyperplane is called the maximum margin hyperplane. In particular, the maximum margin hyperplane is the one that maximizes the quantity M such that, for each positive training observation (x₁, . . . , xₚ), β₀+β₁x₁+β₂x₂+ . . . +βₚxₚ≥M and, for each negative training observation (x₁, . . . , xₚ), β₀+β₁x₁+β₂x₂+ . . . +βₚxₚ≤−M. These inequalities are called the “margin constraints.”

Because real data is noisy, it may be desirable to permit some training observations to violate the margin constraints. In particular, the margin M in the margin constraint for the ith training observation can be weakened to M(1−ϵᵢ), where ϵᵢ is called a “slack” variable. To limit the extent to which the training observations violate the margin constraints, an additional constraint requires that the slack variables sum to at most C, where C is a tuning parameter.

Hence, training an SVC involves solving for the values β₀ through βₚ, ϵ₁ through ϵₙ, and M in Equations 3-6:

$${Maximize}_{{β}_{0} , {β}_{1} , … , {β}_{p} , {ϵ}_{1} , … , {ϵ}_{n} , M} M \qquad ( 3 )$$

subject to:

Σⱼ₌₁^{p}βⱼ²=1, (4)

yᵢ(β₀+β₁xᵢ₁+β₂xᵢ₂+ . . . +βₚxᵢₚ)±M(1−ϵᵢ), and (5)

ϵᵢ≥0,Σᵢ₌₁ⁿϵᵢ≤C (6)

In Equations 3-6, n is the number of training observations, and p is the dimensionality of each training observation. For example, if each training observation consists of 60 data points (e.g., measured breathing rates and gait speed for 30 consecutive previous days), p=60. The value xᵢⱼ in Equation 5 represents the jth data point in the ith training observation, and the value yᵢ represents the ith ground-truth inflammation label (e.g., as determined by an F.Cal measurement), where yᵢ is 1 if the ith observation is positive and −1 if it is negative.

An SVM is a generalization of an SVC to allow for non-linear boundaries between two classes (e.g., between inflammation and non-inflammation). Non-linear boundaries are introduced by increasing the dimensionality of the input. For example, the dimensionality could be increased from p=60 to p=120 by including, for each value xᵢ, its square xᵢ² as an additional input, for example, to train an SVM according to Equations 7-11:

$${Maximize}_{{β}_{0} , {β}_{11} , {β}_{12} , … , {β}_{{p}_{1}} , {β}_{{p}_{2}} , {ϵ}_{1} , … , {ϵ}_{n} , M} M \qquad ( 7 )$$

subject to:

yᵢ(β₀+Σⱼ₌₁^{p}βⱼ₁xᵢⱼ+Σⱼ₌₁^{p}βⱼ₂xᵢⱼ²)≥M(1−ϵᵢ), (8)

Σᵢ₌₁ⁿϵᵢ≤C, (9)

ϵᵢ≥0, and (10)

Σⱼ₌₁^{p}Σₖ₌₁²βⱼₖ²=1 (11)

In these equations, n is the number of training observations, p is the dimensionality of each training observation, β₀, β₁₁, β₁₂, . . . , βₚ₁, βₚ₂ is a set of weights to be learned, ϵ₁, . . . , ϵₙ is a set of slack variables to be learned, C is a tuning parameter, and M is a margin to be maximized.

As in the case of an SVC, an SVM is trained by providing it with a set of training observations X and ground truth labels Y.

In an alternative embodiment, a neural network can take as input the raw reflected RF signals represented as a complex 3-dimensional tensor, where the dimensions are time, x, and y, where x and y represent a location in space, or alternatively the dimensions can be time, angle, and distance. The values of the tensors are the magnitudes and phases of the reflected signals. As in the case of an SVC or SVM, a neural network is trained to classify each test observation as either positive or negative, e.g., as either indicating inflammation or not. One can train a neural-network classifier with the same ground truth labels Y as above, where the goal is for the neural network to predict the same flare labels (e.g., inflammation or no inflammation/remission) as in the SVC/SVM approach. The advantage of neural networks is that the quantifiable health metric(s) is/are not needed, which can make the model more generalizable and allow us to capture metrics that were not explicitly envisioned and specified by the model creator. However, more data may be required to successfully train the neural network to achieve high accuracy.

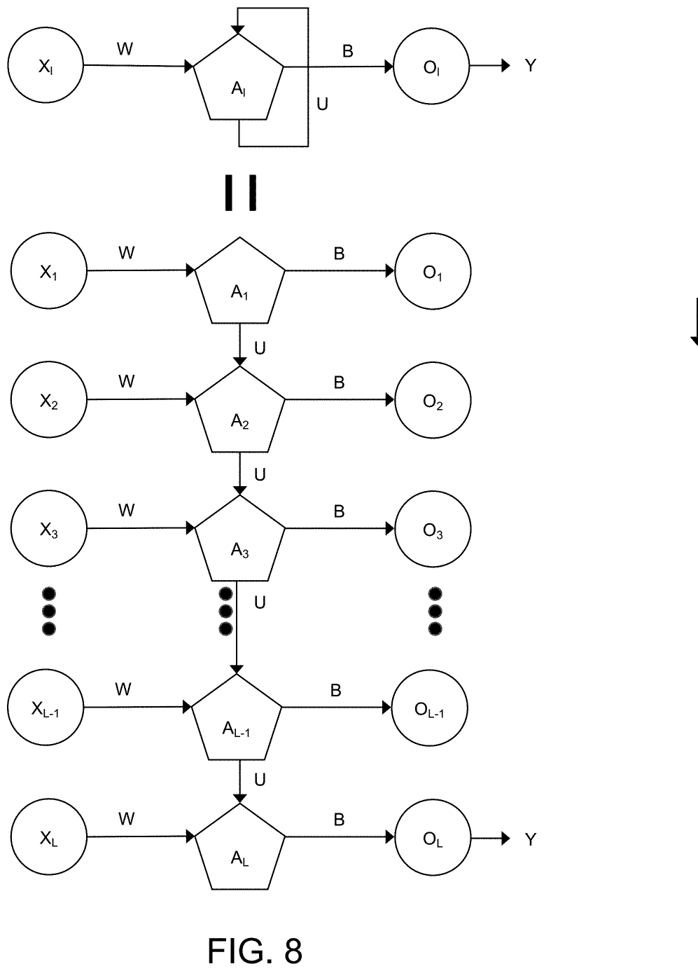

As an example, a neural network such as a recurrent neural network (RNN) can be trained for use as a classifier. When used as a classifier, the input to the RNN is a sequence of values ordered in time (e.g., RF signals collected over the past 30 days) fed to the network one after another. The output is a classification, e.g., inflammation or no inflammation. The structure of an RNN with one hidden layer is illustrated in FIG. 8. The input is a sequence of elements X₁ though X_{L}, representing, e.g., the measured radio signals at times 1 through L. The output O_{L} indicates whether inflammation is detected at time L based on X₁ though X_{L}. W, U, and B in FIG. 8 are weights. The network has a hidden layer A that is described by Equation 12:

[symbol]

=g(wₖ₀+Σⱼ₌₁^{p} [symbol] +Σₛ₌₁^{K} [symbol] ) (12)

In Equation 12, [symbol] denotes the hidden layer A after [symbol] elements have been input to the network. The hidden layer A has k units (i.e., hidden variables), with [symbol] through [symbol] representing the values of these variables after [symbol] elements have been input. Each of the input elements [symbol] has p components [symbol] through [symbol] where a component might be a complex number (magnitude and phase) representing the radio reflection at time [symbol] at a specific angle and distance or at a particular x,y coordinate in space. The function g is a non-linear activation function, e.g., a sigmoid or a ReLU (rectified linear unit), as shown in Equations 13 and 14, respectively. The output classification after [symbol] elements have been input, [symbol] is provided in Equation 15.

$$g ( z ) = \frac{{e}^{z}}{1 + {e}^{z}} = \frac{1}{1 + {e}^{- z}} \qquad ( 13 )$$

$$g ( z ) = {( z )}_{+} = \{ \begin{matrix}0 & if z < 0 \\ z & otherwise\end{matrix} \qquad ( 14 )$$

$${O}_{ℓ} = {β}_{0} + {\sum }_{k = 1}^{K} {β}_{k} {A}_{ℓ k} \qquad ( 15 )$$

Training the RNN includes finding a set of values for the weights wₖ₀ through wₖₚ, and [symbol] through [symbol] in Equation 12 and weights β₀ through β_{K} in Equation 15 that minimizes the error that the network makes in classifying a set of n training observations where ground truth labels are known (e.g., from F.Cal measurements of inflammation). Each of the n training observations includes a sequence of L elements X₁ though X_{L} and a ground truth label Y. The weights are chosen, e.g., to minimize the value in Equation 16, which is the error:

Σᵢ₌₁ⁿ(yᵢ−O_{iL})²=Σᵢ₌₁ⁿ(yᵢ−(β₀+Σₖ₌₁^{K}βₖg(wₖ₀+Σⱼ₌₁^{p}wₖⱼx_{iLj}+Σₛ₌₁^{K}uₖₛa_{i,L-1,x})))² (16)

In Equation 16, yᵢ is the ground truth label for the ith training observation, and O_{iL} is the output after the Lth element in the ith training observation has been input. On the right side of Equation 16, lowercase letters are used for variables whose values depend on a specific training observation. Thus, x_{iLj} is the value of the jth component of the Lth input element in the ith training observation, and a_{i,L-1,s} is the value of the sth hidden variable after L−1 elements in the ith training observation have been input.

Once the RNN classifier has been trained, a new sequence of measurements X₁* through X_{L}* can be classified as either inflammation or not by feeding them as inputs to the RNN in order and examining the value of the output O_{L}. If O_{L} is positive then the classification at time L is positive, otherwise it is negative.

Additionally or alternatively, the RNN may have more than one hidden layer, and an RNN with many hidden layers is called “deep.” The advantage of incorporating multiple layers in a neural network is that while a network with a single layer can be trained to classify as well as a network with multiple layers (a fact known as the “Universal Approximation Theorem”), a network with multiple layers typically requires fewer hidden variables in total. Hence the amount of computation required to train the multi-layer network and to use the multi-layer network to perform classification is less.

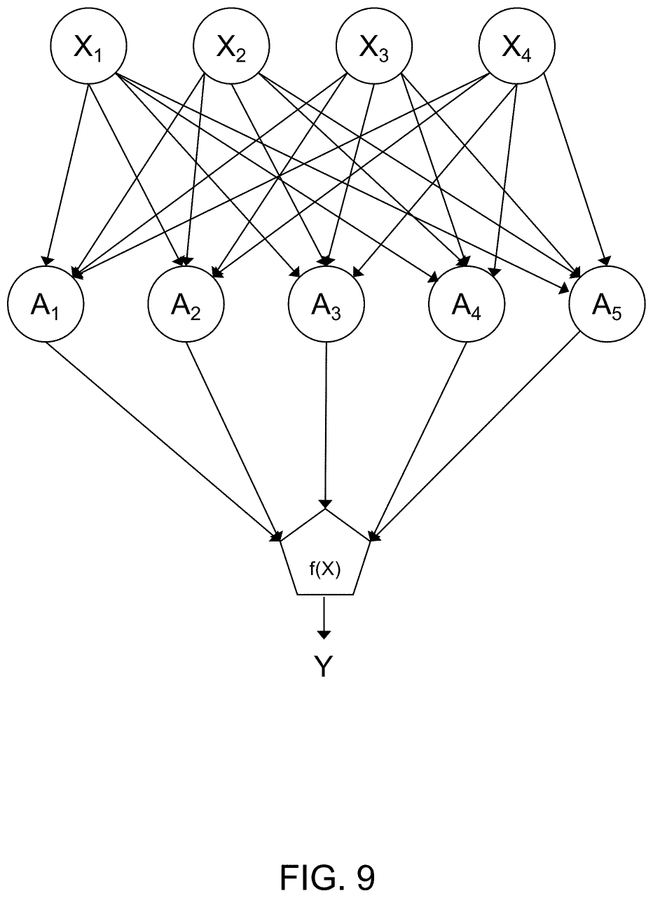

Additionally or alternatively, rather than feeding each input element to an RNN one after another, a sequence of measurements, e.g., the radio measurements collected over 30 consecutive days, might be fed all at once to a feedforward neural network (FNN). An FNN has an input layer, zero or more hidden layers, and an output layer. An example illustration of the structure of an FNN with a single hidden layer is shown in FIG. 9. The input elements X in our example represent the reflections of the radio signals at different times from different points in space. Each input element is a complex number representing the reflection at a certain point in time from a certain angle and distance or x, y position in space. The hidden layer Aₖ is described in Equation 17, and the output layer f(X) is described in Equation 18. In Equation 17, Aₖ is the kth hidden variable, wₖ₀ through wₖₚ are weights, Xⱼ is one of p input elements from some time and position in space, and g is a non-linear function as in Equation 13 or 14. In Equation 18, f(X) is the output and β₀ through βₖ are weights.

Aₖ=g(wₖ₀+Σⱼ₌₁^{p}wₖⱼXⱼ) (17)

f(X)=β₀+Σₖ₌₁^{K}βₖAₖ (18)

Given a set of n training observations, each of which includes p input elements, an FNN can be trained by minimizing the classification error on the ground-truth labels. The goal of training the FNN is to find values for the weights wₖ₀ through wₖₚ in Equation 17 and for the weights β₀ through β_{K} in Equation 19 that minimizes the total error, e.g., that minimizes the squared-error loss quantity shown in Equation 19.

Σᵢ₌₁ⁿ(yᵢ−f(xᵢ))² (19)

Once the FNN classifier has been trained, a new sequence of measurements X can be classified as either inflammation or not by feeding them, all at once, as inputs to the FNN and examining the value of the output f(X). If f(X) is positive, then the observation is positive (e.g., inflammation is present). Otherwise the observation is negative.

Additionally or alternatively, an FNN may have more than one hidden layer. An FNN with multiple hidden layers may require fewer hidden variables and hence may require less computation to train and to use as a classifier. Increasing the number of layers in a neural network is straightforward. To add a second hidden layer to the network described in Equations 17 and 18 for example, the hidden variables Aₖ are replaced by two layers of hidden variables Aₖ⁽¹⁾ and Aₗ⁽²⁾, where the superscripts indicate the layer number. The index k takes on values 1, . . . , K₁ and the index l takes on values 1, . . . , K₂, i.e., there are K₁ hidden variables in the first layer and K₂ hidden variables in the second layer. The input elements X are used as inputs to the first hidden layer. The values of the hidden variables Aₖ⁽¹⁾ in the first hidden layer are then used as inputs to the second hidden layer. The output f(X) is a function of the hidden variables Aₗ⁽²⁾ in the second hidden layer. The equations for the hidden variable and output are given by Equations 20 through 22 below:

Aₖ⁽¹⁾=g(wₖ₀⁽¹⁾+Σⱼ₌₁^{p}wₖⱼ⁽¹⁾Xⱼ) (20)

Aₗ⁽²⁾=g(wₗ₀⁽²⁾+Σₖ₌₁^{K}^{₁}wₗₖ⁽²⁾Aₖ⁽¹⁾) (21)

f(X)=β₀+Σₗ₌₁^{K}^{₂}βₗAₗ⁽²⁾ (22)

In these equations, g is a non-linear function, e.g., as in Equation 13 or 14. Training the 2-layer network includes finding values for (a) the weights wₖⱼ⁽¹⁾, where k runs from 0 to K₁ and j runs from 1 to p, (b) the weights wₗₖ⁽²⁾, where l runs from 0 to K₂ and k runs from 1 to K₁, and (c) the weights βₗ where l runs from 0 to K₂. As in the case of training a one-layer network, the goal is to minimize the loss, e.g., as specified in Equation 19.

Additional hidden layers may be added in an analogous fashion. Furthermore, the same approach can be used to add additional hidden layers to as in an RNN.

Additionally or alternatively, rather than using the value −1 to indicate a negative ground truth label, a value of 0 could be used instead. In this case, the output of an RNN or FNN can be interpreted as the probability that inflammation has occurred, or the level of confidence that the model has that inflammation has occurred. An observation is classified as positive (i.e., inflammation has occurred) if this probability or confidence exceeds a fixed threshold, e.g., if it is larger than ½.

Additionally or alternatively, the FNN may be or include a convolutional neural network (CNN). A convolutional neural network is composed of alternating “convolution” and “pooling” layers. A convolution layer includes one or more “convolution filters.” A convolution filter is defined by a small set of learned weights. As a simplified example, suppose that the input elements for time step 1 are X₁ through X₁₂, and these elements cover a 4-by-3 two-dimensional space of pixels, organized as a two-dimensional array:

| | | |
|---|---|---|
| X₁ | X₂ | X₃ |
| X₄ | X₅ | X₆ |
| X₇ | X₈ | X₉ |
| X₁₀ | X₁₁ | X₁₂ |

In this example, the convolution filter includes or consists of a set of 4 weights to be learned, w₁ through w₄, organized as follows:

| | |
|---|---|
| w₁ | w₂ |
| w₃ | w₄ |

The filter is applied to the 4×3 array of input elements. In particular, in this example, for input variables X₁ through X₁₂, there are six hidden variables A₁⁽¹⁾ through A₆⁽¹⁾, computed as follows:

A₁⁽¹⁾=g(w₁X₁+w₂X₂+w₃X₄+w₄X₅) (23)

A₂²=g(w₁X₂+w₂X₃+w₃X₅+w₄X₆) (24)

A₃⁽¹⁾=g(w₁X₄+w₂X₅+w₃X₇+w₄X₈) (25)

A₄⁽¹⁾=g(w₁X₅+w₂X₆+w₃X₈+w₄X₉) (26)

A₅⁽¹⁾=g(w₁X₇+w₂X₈+w₃X₁₀+w₄X₁₁) (27)

A₆⁽¹⁾=g(w₁X₈+w₂X₉+w₃X₁₁+w₄X₁₂) (28)

In Equations 23-28, g is a non-linear function such as the ReLU function of Equation 14.

In the convolution layer, the same convolution filter is applied to every other 4×3 grouping of inputs, e.g., to the input variables for time step 2, X₁₃ through X₂₄. A convolution layer may include more than one convolution filter. Each convolution filter is defined by its own set of weights and is applied to each grouping of inputs, producing its own set of hidden variables.

Because each hidden variable depends on only a small number of input elements or hidden variables at the previous layer, and because, for each convolution filter, the same small set of weights is used to compute each hidden variable, the computational effort required to train a CNN (i.e., to learn the weights) is reduced compared to an FNN in which every hidden variable at one layer depends on every input element or hidden variable at the previous layer.

In a pooling layer, the non-linear function g of Equations 13 and 14, which takes a single input, is replaced by a function g of several inputs. Most commonly the maximum function is used, and in this case the pooling layer is called a “max-pooling layer.” As in a convolution layer, each hidden variable in a pooling layer typically depends on only a subset of the inputs or hidden variables in the previous layer. Continuing with the simplified example above, two hidden variables A₁⁽²⁾ and A₂⁽²⁾ in a pooling layer that follows the convolution layer could be defined as:

A₁⁽²⁾=max(A₁⁽¹⁾,A₂⁽¹⁾,A₃⁽¹⁾,A₄⁽¹⁾) (29)

A₂⁽²⁾=max(A₃⁽¹⁾,A₄⁽¹⁾,A₅⁽¹⁾,A₆⁽¹⁾) (30)

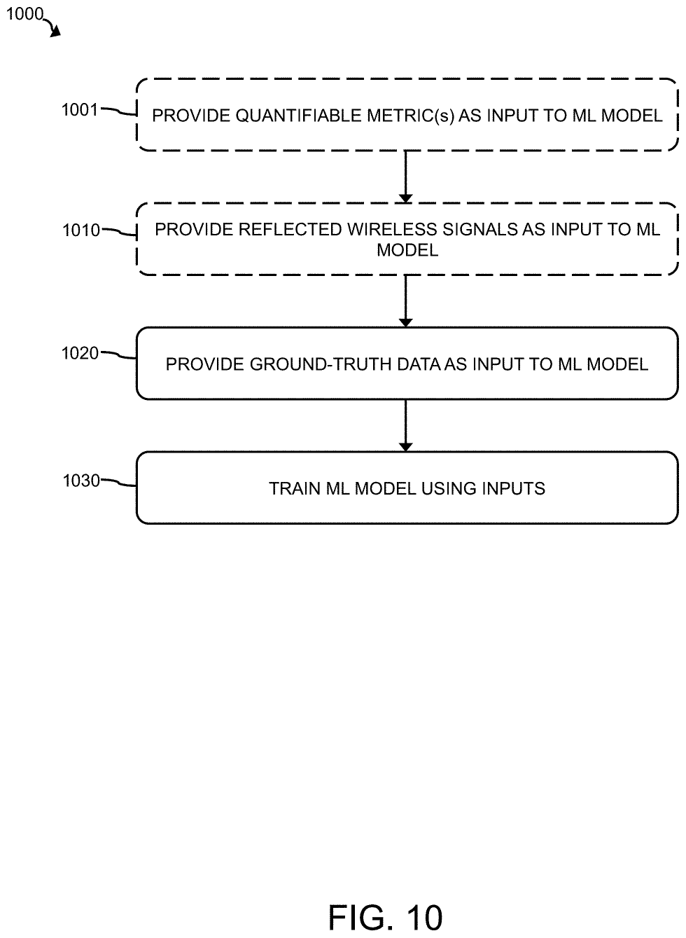

FIG. 10 is a flow chart of a method 1000 for training an ML model according to an embodiment. The ML model can include a ML classifier (e.g., an SVM, an SVC, or another ML classifier), a neural network, an RNN, an FNN, a CNN, and/or another ML model.

In optional step 1001, ground-truth data representing one or more quantifiable health metrics of one or more subjects with respect to time is/are provided as inputs to an untrained ML model. The quantifiable health metrics can include the subject's breathing rate, the sleep stages, and/or the gait speed. The quantifiable health metrics can also include features or statistics relating to the quantifiable health metrics such as the average and/or median breathing rate, the length and/or percentage of time in each sleep stage, the average and/or median gait speed, and/or other features or statistics. The quantifiable health metric data includes the time and date for each quantifiable health metric data value.

In optional step 1010, ground-truth reflected FMCW wireless data of one or more subjects with respect to time is provided as an input to the untrained ML model. The ground-truth reflected FMCW wireless data can include ground-truth raw reflected FMCW wireless data and/or ground-truth three-dimensional reflected wireless maps. The ground-truth reflected FMCW wireless data includes the time and date when the respective data is measured/collected.

In step 1020, ground-truth data is provided as an input to the untrained ML model. The ground-truth data can include the F.Cal measurements of the subject. Additionally or alternatively, the ground-truth data can include labels or digital representations (e.g., a “1” or “−1”) of labels that indicate whether the subject is in an inflammatory or a non-inflammatory (e.g., remission) state and the respective dates of the labels and/or F.Cal measurements. It is noted that step 1001, optional step 1010, and step 1020 can occur in any order or two more steps can occur simultaneously.

In step 1030, the untrained ML model is trained (e.g., as described herein) using the inputs in optional step 1001, in optional step 1010, and in step 1020. In some embodiments, the untrained ML model (e.g., an untrained ML classifier or an untrained RNN, FNN, or CNN) can be trained using the inputs in optional step 1001 but not the inputs in optional step 1010. In other embodiments, the untrained ML model (e.g., an untrained RNN, FNN, or CNN) can be trained using with the inputs in optional step 1010 but not the inputs in optional step 1001. In other embodiments, the untrained ML model (e.g., an untrained ML classifier) can be trained using with the inputs in optional step 1001 and the inputs in optional step 1010.

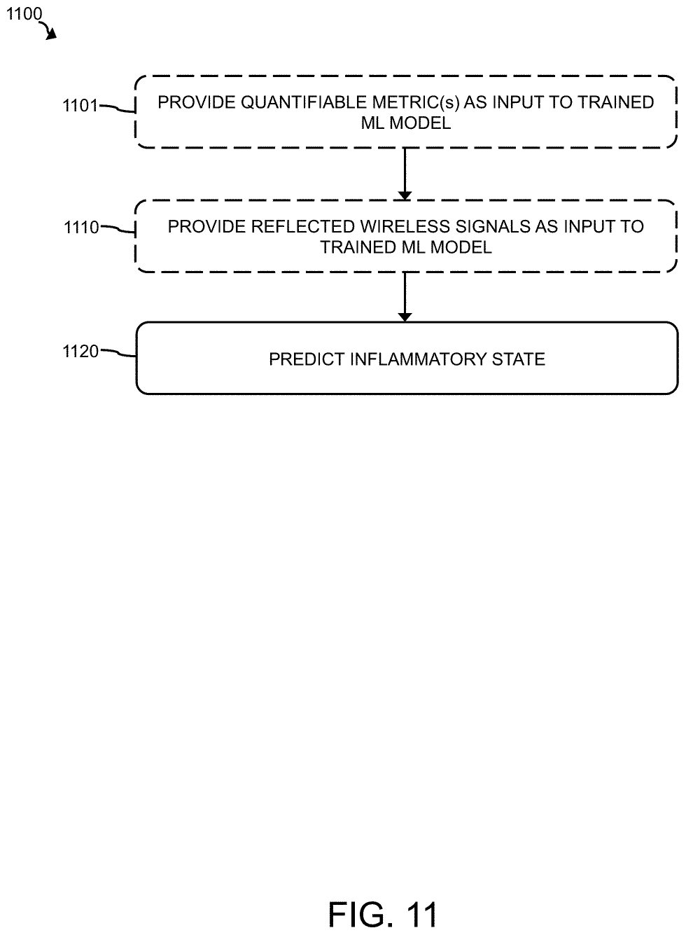

FIG. 11 is a flow chart of a computer-implemented method 1100 for predicting the inflammatory state of a subject according to an embodiment. Step 650 (FIG. 6) can be performed using method 1100.

In optional step 1101, one or more quantifiable health metrics is/are provided as inputs to a trained ML model. The trained ML model can include a trained ML classifier (e.g., a trained SVM, a trained SVC, or another trained ML classifier), a trained neural network, a trained RNN, and/or a trained FNN. The trained ML model can be trained according to method 1000. The quantifiable health metrics input in step 1101 are preferably the same type of quantifiable health metrics used to train the ML model in step 1001 (FIG. 10). For example, when the average gait speed is provided as an input to train the ML classifier in step 1001, the average gait speed is preferably used as an input to the trained ML classifier in step 1101. The quantifiable health metric data includes the time and date for each quantifiable health metric data value.

In optional step 1110, raw reflected RF signals (e.g., reflected FMCW wireless signals) are provided as an input to the trained ML model. The raw reflected RF data includes the time and date when each raw reflected RF signal is measured/collected. The raw reflected RF data can include heatmaps that include an array of voxels with each voxel corresponding to a respective physical location in the room (or other location) in which the person under observation is located. The numerical value, in real or complex form, of each voxel represents the magnitude and phase of the wireless signal reflected from the respective/corresponding physical location.

In step 1120, the trained ML classifier predicts, using the inputs provided in step 1101 and/or step 1110, the inflammatory state of the subject. The predicted inflammatory state is either a predicted inflammatory state or a predicted non-inflammatory (e.g., remission) state.

Steps 1101-1120 can be repeated as additional data is collected. For examples, steps 1101-1120 can be repeated continuously, periodically (e.g., hourly, daily, or another period), irregularly, or on another basis.

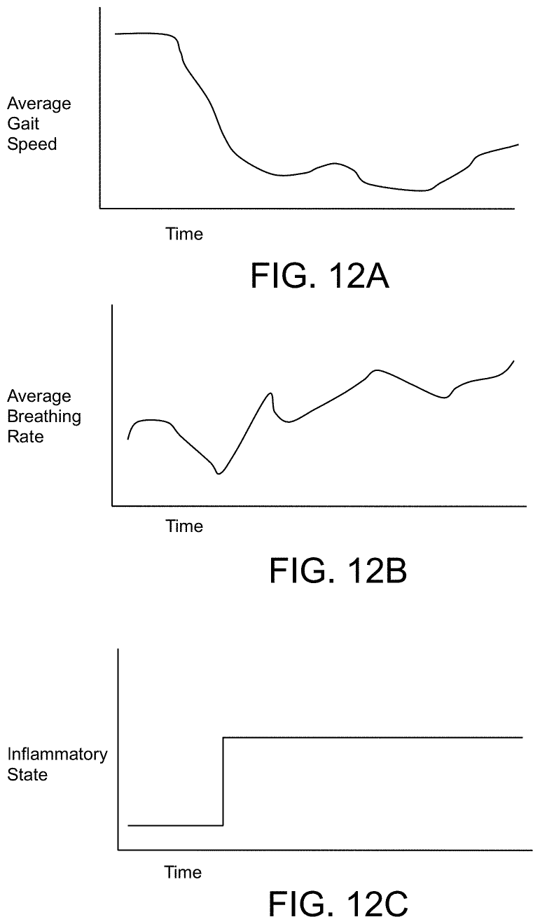

FIGS. 12A and 12B are example graphs that illustrate the subject's average gait speed and average breathing rate, respectively, with respect to time. FIGS. 12A and 12B can represent the quantifiable health metric data provided as an input to the untrained ML model in step 1001 (FIG. 10) or to the trained ML model in step 1101 (FIG. 11). FIG. 12C is an example graph that illustrates the predicted inflammatory state of the subject, for example as predicted by the trained ML model in step 1120 (FIG. 11). The inflammatory state is predicted to be high (in an inflamed state) at the time when the average gate speed decreases and the average breathing rate increases.

The invention should not be considered limited to the particular embodiments described above. Various modifications, equivalent processes, as well as numerous structures to which the invention may be applicable, will be readily apparent to those skilled in the art to which the invention is directed upon review of this disclosure. The above-described embodiments may be implemented in numerous ways. One or more aspects and embodiments involving the performance of processes or methods may utilize program instructions executable by a device (e.g., a computer, a processor, or other device) to perform, or control performance of, the processes or methods.

In this respect, various inventive concepts may be embodied as a non-transitory computer readable storage medium (or multiple non-transitory computer readable storage media) (e.g., a computer memory of any suitable type including transitory or non-transitory digital storage units, circuit configurations in Field Programmable Gate Arrays or other semiconductor devices, or other tangible computer storage medium) encoded with one or more programs that, when executed on one or more computers or other processors, perform methods that implement one or more of the various embodiments described above. When implemented in software (e.g., as an app), the software code may be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more communication devices, which may be used to interconnect the computer to one or more other devices and/or systems, such as, for example, one or more networks in any suitable form, including a local area network or a wide area network, such as an enterprise network, and intelligent network (IN) or the Internet. Such networks may be based on any suitable technology and may operate according to any suitable protocol and may include wireless networks or wired networks.

Also, a computer may have one or more input devices and/or one or more output devices. These devices can be used, among other things, to present a user interface. Examples of output devices that may be used to provide a user interface include printers or display screens for visual presentation of output and speakers or other sound generating devices for audible presentation of output. Examples of input devices that may be used for a user interface include keyboards, and pointing devices, such as mice, touch pads, and digitizing tablets. As another example, a computer may receive input information through speech recognition or in other audible formats.

The non-transitory computer readable medium or media may be transportable, such that the program or programs stored thereon may be loaded onto one or more different computers or other processors to implement various one or more of the aspects described above. In some embodiments, computer readable media may be non-transitory media.

The terms “program,” “app,” and “software” are used herein in a generic sense to refer to any type of computer code or set of computer-executable instructions that may be employed to program a computer or other processor to implement various aspects as described above. Additionally, it should be appreciated that, according to one aspect, one or more computer programs that when executed perform methods of this application need not reside on a single computer or processor but may be distributed in a modular fashion among a number of different computers or processors to implement various aspects of this application.

Computer-executable instructions may be in many forms, such as program modules, executed by one or more computers or other devices. Generally, program modules include routines, programs, objects, components, data structures, etc. that performs particular tasks or implement particular abstract data types. The functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Thus, the disclosure and claims include new and novel improvements to existing methods and technologies, which were not previously known nor implemented to achieve the useful results described above. Users of the method and system will reap tangible benefits from the functions now made possible on account of the specific modifications described herein causing the effects in the system and its outputs to its users. It is expected that significantly improved operations can be achieved upon implementation of the claimed invention, using the technical components recited herein.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

What is claimed is:

1. A wireless method for predicting an inflammation state of a person under observation, comprising:
(a) transmitting frequency-modulated continuous-wave (FMCW) wireless signals from one or more transmitting antennas;
(b) receiving reflected FMCW wireless signals with one or more receiving antennas, at least some of the reflected FMCW wireless signals being reflected from the person partially or fully;
(c) repeating steps (a) and (b) continuously while the person is under observation;
(d) producing reflected FMCW wireless data based on the reflected FMCW wireless signals;
(e) providing the reflected FMCW wireless data as an input to a trained machine-learning (ML) model, the trained ML model having been trained with ground-truth inflammation that represents ground-truth inflammation states of one or more subjects with respect to time and with ground-truth reflected FMCW wireless data of the one or more subjects with respect to time; and
(f) predicting, with the trained ML model, whether the person under observation is in an inflamed state or in a non-inflamed state.

2. The method of claim 1, wherein the trained ML model includes a trained neural network.

3. The method of claim 2, wherein the trained ML model includes a trained recurrent neural network, a trained feedforward neural network, or a trained convolutional neural network.

4. The method of claim 1, wherein:
step (d) comprises converting a discrete time period of raw reflected FMCW wireless data into a three-dimensional reflected wireless signal map, the three-dimensional reflected wireless signal map including a plurality of voxels that correspond to a respective physical location in a room in which the person is located, each voxel having a respective numerical value that represents a magnitude and a phase of the reflected FMCW wireless signal(s) that was/were reflected from the respective physical location, and
step (e) comprises providing the three-dimensional reflected wireless signal map as the input to the trained ML model, the trained ML model having been trained with ground-truth three-dimensional reflected wireless signal maps with respect to time.

5. The method of claim 4, wherein:
the input to the trained ML model further includes the raw reflected FMCW wireless data, and
the trained ML model was trained with ground-truth raw reflected FMCW wireless data with respect to time.

6. A wireless method for predicting an inflammation state of a person under observation, comprising:
(a) transmitting frequency-modulated continuous-wave (FMCW) wireless signals from one or more transmitting antennas;
(b) receiving reflected FMCW wireless signals with one or more receiving antennas, at least some of the reflected FMCW wireless signals being reflected from the person partially or fully;
(c) repeating steps (a) and (b) continuously while the person is under observation;
(d) producing raw reflected FMCW wireless data from the reflected FMCW wireless signals;
(e) converting a plurality of discrete time periods of the raw reflected FMCW wireless data into respective three-dimensional reflected wireless signal maps, each three-dimensional reflected wireless signal map including a plurality of voxels that correspond to a respective physical location in a room in which the person under observation is located, each voxel having a respective numerical value that represents a magnitude and a phase of the reflected FMCW wireless signal(s) that was/were reflected from the respective physical location;
(f) determining a health indicator of the person under observation based on a plurality of three-dimensional reflected wireless signal maps;
(g) determining one or more quantifiable health metrics related to the health indicator;
(h) providing the quantifiable health metric(s) as an input to a trained machine-learning (ML) model, the trained ML model having been trained with ground-truth inflammation that represents ground-truth inflammation states of one or more subjects with respect to time and with ground-truth quantifiable health metric data of the one or more subjects with respect to time; and
(i) predicting, with the trained ML model, whether the person under observation is in an inflamed state or in a non-inflamed state.

7. The method of claim 6, wherein the trained ML model includes a trained ML classifier.

8. The method of claim 7, wherein the trained ML classifier includes a support vector classifier or a support vector machine.

9. The method of claim 6, wherein:
the input to the trained ML model further includes the respective three-dimensional reflected wireless signal maps, and
the trained ML model was trained with ground-truth three-dimensional reflected wireless signal maps with respect to time.

10. The method of claim 9, wherein:
the input to the trained ML model further includes the raw reflected FMCW wireless data, and
the trained ML model was trained with ground-truth raw reflected FMCW wireless data with respect to time.

11. The method of claim 6, wherein:
the health indicator includes a respiration of the subject under observation,
the quantifiable health metric(s) include a respiration rate of the subject under observation and/or an average respiration rate of the subject under observation, and
the ground-truth quantifiable health metric data includes a ground-truth respiration rate of the one or more subjects with respect to time and/or an average ground-truth respiration rate of the one or more subjects with respect to time.

12. The method of claim 11, wherein:
the health indicator is a first health indicator,
the quantifiable health metric(s) is/are first quantifiable health metric(s), and
the method further comprises:
determining a second health indicator of the person under observation based on the three-dimensional wireless signal maps;
determining one or more second quantifiable health metrics related to the second health indicator; and
providing the first and second quantifiable health metric(s) as the input to the trained ML model, wherein the ground-truth quantifiable health metric data used to train the trained ML model is related to the first and second quantifiable health metrics.

13. The method of claim 12, wherein:
the second health indicator includes a physical location of the subject under observation,
the second quantifiable health metric(s) include a gate speed of the subject under observation and/or an average gate speed of the subject under observation, and
the ground-truth quantifiable health metric data further includes a ground-truth gate speed of the one or more subjects with respect to time and/or an average gate speed of the one or more subjects with respect to time.

14. The method of claim 6, further comprising, sending an output signal to a device or an account controlled by the subject under observation, the output signal including whether the person under observation is in the inflamed state or in the non-inflamed state.

15. A wireless-tracking system comprising:
one or more transmitting antennas configured to transmit frequency-modulated continuous-wave (FMCW) wireless signals;
one or more receiving antennas configured to receive reflected FMCW wireless signals, at least some of the reflected FMCW wireless signals being reflected, partially or fully, from a person under observation;
a processor circuit electrically coupled to the one or more transmitting antennas and the one or more receiving antennas;
a power supply electrically coupled to the processor circuit; and
non-transitory computer-readable memory in electrical communication with the processor circuit, the non-transitory computer-readable memory storing computer-readable instructions that, when executed by the processor circuit, cause the processor circuit to:
produce reflected FMCW wireless data based on the reflected FMCW wireless signals;
provide the reflected FMCW wireless data as an input to a trained machine-learning (ML) model, the trained ML model having been trained with ground-truth inflammation that represents ground-truth inflammation states of one or more subjects with respect to time and with ground-truth reflected FMCW wireless data of the one or more subjects with respect to time; and
predict, with the trained ML model, whether the person under observation is in an inflamed state or in a non-inflamed state.

16. The system of claim 15, further comprising:
the one or more transmitting antennas comprise a plurality of the transmitting antennas,
the one or more receiving antennas comprise a plurality of the receiving antennas, and
the transmitting and receiving antennas are arranged along two orthogonal axes.

17. The system of claim 16, wherein the transmitting antennas and the receiving antennas are evenly spaced along the two orthogonal axes.

18. The system of claim 15, wherein the computer-readable instructions that, when executed by the processor circuit, further cause the processor circuit to:
convert a discrete time period of raw reflected FMCW wireless data into a three-dimensional reflected wireless signal map, the three-dimensional reflected wireless signal map including a plurality of voxels that correspond to a respective physical location in a room in which the person is located, each voxel having a respective numerical value that represents a magnitude and a phase of the reflected FMCW wireless signal(s) that was/were reflected from the respective physical location, and
providing the three-dimensional reflected wireless signal map as the input to the trained ML model, the trained ML model having been trained with ground-truth three-dimensional wireless signal maps with respect to time.

19. A system for determining an inflammation state of a person under observation, comprising:
a wireless-tracking system comprising:
one or more transmitting antennas configured to transmit frequency-modulated continuous-wave (FMCW) wireless signals;
one or more receiving antennas configured to receive reflected FMCW wireless signals, at least some of the reflected FMCW wireless signals being reflected, partially or fully, from a person under observation;
a first processor circuit electrically coupled to the one or more transmitting antennas and the one or more receiving antennas;
a power supply electrically coupled to the first processor circuit; and
a first non-transitory computer-readable memory in electrical communication with the first processor circuit, the first non-transitory computer-readable memory storing computer-readable instructions that, when executed by the first processor circuit, cause the first processor circuit to:
produce reflected FMCW wireless data based on the reflected FMCW wireless signals; and
send the reflected FMCW wireless data to a computer,
wherein the computer comprises:
a second processor circuit;
a second non-transitory computer-readable memory in electrical communication with the second processor circuit, the second non-transitory computer-readable memory storing computer-readable instructions that, when executed by the second processor circuit, cause the second processor circuit to:
store the reflected FMCW wireless data in the second non-transitory computer-readable memory;
provide the reflected FMCW wireless data as an input to a trained machine-learning (ML) model, the trained ML model having been trained with ground-truth inflammation that represents ground-truth inflammation states of one or more subjects with respect to time and with ground-truth reflected FMCW wireless data of the one or more subjects with respect to time; and
predict, with the trained ML model, whether the person under observation is in an inflamed state or in a non-inflamed state.

20. The system of claim 19, wherein:
the computer-readable instructions stored on the first non-transitory computer-readable memory, when executed by the first processor circuit, cause the first processor circuit to:
convert a plurality of discrete time periods of raw reflected FMCW wireless data into respective three-dimensional reflected wireless signal maps, each three-dimensional reflected wireless signal map including a plurality of voxels that correspond to a respective physical location in a room in which the person under observation is located, each voxel having a respective numerical value that represents a magnitude and a phase of the reflected FMCW wireless signal(s) that was/were reflected from the respective physical location; and
send three-dimensional reflected wireless signal maps to the computer, and
the computer-readable instructions stored on the second non-transitory computer-readable memory, when executed by the second processor circuit, cause the second processor circuit to:
store the three-dimensional reflected wireless signal maps in the second non-transitory computer-readable memory;
determine a health indicator of the person under observation based on a plurality of the three-dimensional reflected wireless signal maps;
determine one or more quantifiable health metrics related to the health indicator; and
provide the quantifiable health metric(s) as the input the trained ML model, the trained ML model having been trained with ground-truth inflammation that represents ground-truth quantifiable health metric data of the one or more subjects with respect to time.
